SEQUENCE LISTING (24 sequences)

SEQ ID NO: 1 - protein, 248 aa, Homo sapiens
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 2 - protein, 228 aa, Homo sapiens
ETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 3 - protein, 141 aa, Homo sapiens
AASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 4 - protein, 115 aa, Homo sapiens
VGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 5 - protein, 148 aa, Homo sapiens
PDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 6 - protein, 381 aa, Artificial Sequence
  Recombinantly-produced fusion protein
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEINITFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 7 - protein, 383 aa, Artificial Sequence
  Recombinantly-produced fusion protein
AKTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 8 - protein, 352 aa, Artificial Sequence
  Recombinantly-produced fusion protein
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGATSKQVGNKFFLTNGEEVITFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 9 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 10 - protein, 10 aa, Homo sapiens
NEGEPNNAGS

SEQ ID NO: 11 - protein, 15 aa, Homo sapiens
LLLKNGQWNDVPCST

SEQ ID NO: 12 - protein, 326 aa, Homo sapiens
MELSGATMARGLAVLLVLFLHIKNLPAQAADTCPEVKVVGLEGSDKLTILRGCPGLPGAPGPKGEAGVIGERGERGLPGAPGKAGPVGPKGDRGEKGMRGEKGDAGQSQSCATGPRNCKDLLDRGYFLSGWHTIYLPDCRPLTVLCDMDTDGGGWTVFQRRMDGSVDFYRDWAAYKQGFGSQLGEFWLGNDNIHALTAQGSSELRVDLVDFEGNHQFAKYKSFKVADEAEKYKLVLGAFVGGSAGNSLTGHNNNFFSTKDQDNDVSSSNCAEKFQGAWWYADCHASNLNGLYLMGPHESYANGINWSAAKGYKYSYKVSEMKVRPA

SEQ ID NO: 13 - protein, 313 aa, Homo sapiens
MELDRAVGVLGAATLLLSFLGMAWALQAADTCPEVKMVGLEGSDKLTILRGCPGLPGAPGPKGEAGTNGKRGERGPPGPPGKAGPPGPNGAPGEPQPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKYRSFKVADEAEKYNLVLGAFVEGSAGDSLTFHNNQSFSTKDQDNDLNTGNCAVMFQGAWWYKNCHVSNLNGRYLRGTHGSFANGINWKSGKGYNYSYKVSEMKVRPA

SEQ ID NO: 14 - protein, 299 aa, Homo sapiens
MDLLWILPSLWLLLLGGPACLKTQEHPSCPGPRELEASKVVLLPSCPGAPGSPGEKGAPGPQGPPGPPGKMGPKGEPGDPVNLLRCQEGPRNCRELLSQGATLSGWYHLCLPEGRALPVFCDMDTEGGGWLVFQRRQDGSVDFFRSWSSYRAGFGNQESEFWLGNENLHQLTLQGNWELRVELEDFNGNRTFAHYATFRLLGEVDHYQLALGKFSEGTAGDSLSLHSGRPFTTYDADHDSSNSNCAVIVHGAWWYASCYRSNLNGRYAVSEAAAHKYGIDWASGRGVGHPYRRVRMMLR

SEQ ID NO: 15 - protein, 786 aa, Homo sapiens
MTSIFHFAIIFMLILQIRIQLSEESEFLVDRSKNGLIHVPKDLSQKTTILNISQNYISELWTSDILSLSKLRILIISHNRIQYLDISVFKFNQELEYLDLSHNKLVKISCHPTVNLKHLDLSFNAFDALPICKEFGNMSQLKFLGLSTTHLEKSSVLPIAHLNISKVLLVLGETYGEKEDPEGLQDFNTESLHIVFPTNKEFHFILDVSVKTVANLELSNIKCVLEDNKCSYFLSILAKLQTNPKLSNLTLNNIETTWNSFIRILQLVWHTTVWYFSISNVKLQGQLDFRDFDYSGTSLKALSIHQVVSDVFGFPQSYIYEIFSNMNIKNFTVSGTRMVHMLCPSKISPFLHLDFSNNLLTDTVFENCGHLTELETLILQMNQLKELSKIAEMTTQMKSLQQLDISQNSVSYDEKKGDCSWTKSLLSLNMSSNILTDTIFRCLPPRIKVLDLHSNKIKSIPKQVVKLEALQELNVAFNSLTDLPGCGSFSSLSVLIIDHNSVSHPSADFFQSCQKMRSIKAGDNPFQCTCELGEFVKNIDQVSSEVLEGWPDSYKCDYPESYRGTLLKDFHMSELSCNITLLIVTIVATMLVLAVTVTSLCSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 16 - protein, 784 aa, Homo sapiens
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 17 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 18 - protein, 839 aa, Homo sapiens
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 19 - protein, 858 aa, Homo sapiens
MGDHLDLLLGVVLMAGPVFGIPSCSFDGRIAFYRFCNLTQVPQVLNTTERLLLSFNYIRTVTASSFPFLEQLQLLELGSQYTPLTIDKEAFRNLPNLRILDLGSSKIYFLHPDAFQGLFHLFELRLYFCGLSDAVLKDGYFRNLKALTRLDLSKNQIRSLYLHPSFGKLNSLKSIDFSSNQIFLVCEHELEPLQGKTLSFFSLAANSLYSRVSVDWGKCMNPFRNMVLEILDVSGNGWTVDITGNFSNAISKSQAFSLILAHHIMGAGFGFHNIKDPDQNTFAGLARSSVRHLDLSHGFVFSLNSRVFETLKDLKVLNLAYNKINKIADEAFYGLDNLQVLNLSYNLLGELYSSNFYGLPKVAYIDLQKNHIAIIQDQTFKFLEKLQTLDLRDNALTTIHFIPSIPDIFLSGNKLVTLPKINLTANLIHLSENRLENLDILYFLLRVPHLQILILNQNRFSSCSGDQTPSENPSLEQLFLGENMLQLAWETELCWDVFEGLSHLQVLYLNHNYLNSLPPGVFSHLTALRGLSLNSNRLTVLSHNDLPANLEILDISRNQLLAPNPDVFVSLSVLDITHNKFICECELSTFINWLNHTNVTIAGPPADIYCVYPDSFSGVSLFSLSTEGCDEEEVLKSLKFSLFIVCTVTLTLFLMTILTVTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDLQDVGWFLHKLSQQILKKEKEKKKDNNIPLQTVATIS

SEQ ID NO: 20 - protein, 796 aa, Homo sapiens
MTKDKEPIVKSFHFVCLMIIIVGTRIQFSDGNEFAVDKSKRGLIHVPKDLPLKTKVLDMSQNYIAELQVSDMSFLSELTVLRLSHNRIQLLDLSVFKFNQDLEYLDLSHNQLQKISCHPIVSFRHLDLSFNDFKALPICKEFGNLSQLNFLGLSAMKLQKLDLLPIAHLHLSYILLDLRNYYIKENETESLQILNAKTLHLVFHPTSLFAIQVNISVNTLGCLQLTNIKLNDDNCQVFIKFLSELTRGSTLLNFTLNHIETTWKCLVRVFQFLWPKPVEYLNIYNLTIIESIREEDFTYSKTTLKALTIEHITNQVFLFSQTALYTVFSEMNIMMLTISDTPFIHMLCPHAPSTFKFLNFTQNVFTDSIFEKCSTLVKLETLILQKNGLKDLFKVGLMTKDMPSLEILDVSWNSLESGRHKENCTWVESIVVLNLSSNMLTDSVFRCLPPRIKVLDLHSNKIKSVPKQVVKLEALQELNVAFNSLTDLPGCGSFSSLSVLIIDHNSVSHPSADFFQSCQKMRSIKAGDNPFQCTCELREFVKNIDQVSSEVLEGWPDSYKCDYPESYRGSPLKDFHMSELSCNITLLIVTIGATMLVLAVTVTSLCIYLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 21 - protein, 1049 aa, Homo sapiens
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 22 - protein, 1041 aa, Homo sapiens
MENMFLQSSMLTCIFLLISGSCELCAEENFSRSYPCDEKKQNDSVIAECSNRRLQEVPQTVGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFDNASALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 23 - protein, 1032 aa, Homo sapiens
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 24 - protein, 811 aa, Homo sapiens
MRLIRNIYIFCSIVMTAEGDAPELPEERELMTNCSNMSLRKVPADLTPATTTLDLSYNLLFQLQSSDFHSVSKLRVLILCHNRIQQLDLKTFEFNKELRYLDLSNNRLKSVTWYLLAGLRYLDLSFNDFDTMPICEEAGNMSHLEILGLSGAKIQKSDFQKIAHLHLNTVFLGFRTLPHYEEGSLPILNTTKLHIVLPMDTNFWVLLRDGIKTSKILEMTNIDGKSQFVSYEMQRNLSLENAKTSVLLLNKVDLLWDDLFLILQFVWHTSVEHFQIRNVTFGGKAYLDHNSFDYSNTVMRTIKLEHVHFRVFYIQQDKIYLLLTKMDIENLTISNAQMPHMLFPNYPTKFQYLNFANNILTDELFKRTIQLPHLKTLILNGNKLETLSLVSCFANNTPLEHLDLSQNLLQHKNDENCSWPETVVNMNLSYNKLSDSVFRCLPKSIQILDLNNNQIQTVPKETIHLMALRELNIAFNFLTDLPGCSHFSRLSVLNIEMNFILSPSLDFVQSCQEVKTLNAGRNPFRCTCELKNFIQLETYSEVMMVGWSDSYTCEYPLNLRGTRLKDVHLHELSCNTALLIVTIVVIMLVLGLAVAFCCLHFDLPWYLRMLGQCTQTWHRVRKTTQEQLKRNVRFHAFISYSEHDSLWVKNELIPNLEKEDGSILICLYESYFDPGKSISENIVSFIEKSYKSIFVLSPNFVQNEWCHYEFYFAHHNLFHENSDHIILILLEPIPFYCIPTRYHKLKALLEKKAYLEWPKDRRKCGLFWANLRAAINVNVLATREMYELQTFTELNEESRGSTISLMRTDCL

(19) United States
(12) Patent Application Publication
(10) Pub. No.: US 2023/0054472 A1
(43) Pub. Date: Feb. 23, 2023

(54) SAMPLE PREPARATION AND MICROBIAL ANALYSIS

(71) Applicant: Miraki Innovation Think Tank LLC, Cambridge, MA (US)

(72) Inventors: James HILL, Arlington, MA (US); Nisha Veronica VARMA, Brookline, MA (US); Gregory T. MARTIN, Boston, MA (US); Goossen Jan Bernard BOER, Brookline, MA (US); Zhiqian ZHOU, Boston, MA (US); Christopher VELIS, Lexington, MA (US)

(73) Assignee: Miraki Innovation Think Tank LLC, Cambridge, MA (US)

(21) Appl. No.: 17/788,137

(22) PCT Filed: Dec. 23, 2020
(86) PCT No.: PCT/US2020/066923

§ 371 Date: Jun. 22, 2022

Related U.S. Application Data
(60) Provisional application No. 63/083,465, filed on Sep. 25, 2020.
(60) Provisional application No. 62/953,083, filed on Dec. 23, 2019.

(51) Int. Cl. G01N 33/569; G01N 33/543; G01N 33/68

(52) U.S. Cl. CPC G01N 33/569; G01N 33/54326; G01N 33/6848; G01N 2333/4724; G01N 2400/00

(57) ABSTRACT

Methods are described for preparing samples including biological, environmental, and food products for microbial analysis. Microbes and microbe components in the sample can be treated with antimicrobial compounds and a matrix solution to permit fast and accurate characterization using analysis techniques such as matrix-assisted laser desorption/ionization Time-of-Flight mass spectrometry (MALDI-TOF MS).

### SEQUENCE LISTING

A sequence listing in electronic (ASCII text file) format is filed with this application and incorporated herein by reference. The name of the ASCII text file is “2020_3237A_ST25.txt”; the file was created on Dec. 23, 2020; the size of the file is 103 KB.

### FIELD OF THE INVENTION

The present disclosure relates to sample preparation and microbe capture and analysis.

### BACKGROUND

Sepsis is a life-threatening condition that results from microbial infections (e.g., bacterial, viral, parasitic, or fungal) and the body's associated response causing damage to tissues. Sepsis is a major cause of death in American intensive care units. While microbes can directly damage tissues, resulting inflammatory responses can cause further damage and lead to septic shock and death.

Early detection of infection and accurate identification of the infecting microbes are keys to successful treatment as different microbes are most susceptible to different treatments. Patients in septic shock should be treated as soon as possible to derive optimal benefit from antimicrobial therapies.

Rapid testing and identification of microbes have applications in food safety, environmental sampling, and other areas in addition to healthcare. However, means of capturing and rapidly and accurately identifying pathogens and other microbes directly from samples (e.g., body fluids) are lacking.

### SUMMARY

The present invention provides systems and methods for analyzing pathogens and other microbes from samples such as blood or other bodily fluids.

The present invention provides:
(1) A method of preparing a sample for detecting a microbe or microbe components present in the sample, the method comprising adding a substance to a sample suspected of comprising a microbe or microbe components; digesting the sample under conditions promoting digestion of a microbe or microbe components; and optionally contacting the digested microbe or microbe components with a matrix or matrix solution on a target substrate.
(2) The method according to the above (1), wherein the substance comprises one or more of an antimicrobial mixture, an enzyme, a protease, or a carbohydrate-cleaving enzyme.
(3) The method according to the above (2), wherein the protease is trypsin.
(4) The method according to the above (1), wherein the sample is a patient sample and the detecting a microbe or microbe component comprises detecting a microbial infection in a patient.
(5) The method according to the above (1), further comprising, prior to adding the substance, contacting the sample with a microbe targeting molecule bound substrate and isolating from the sample a microbe or microbe components bound to the microbe targeting molecule.
(6) The method according to the above (5), wherein the substrate is a magnetic substrate, a fiber substrate, a polymer substrate, or ELISA plate.
(7) The method according to the above (5), wherein the step of isolating comprises applying a magnet or magnetic field to the sample.
(8) The method according to the above (5), wherein the step of isolating comprises washing the substrate with a fluid to remove unbound cells, biomolecules, or chemicals.
(9) The method according to the above (8), wherein fluid comprises calcium.
(10) The method according to the above (5), wherein the step of isolating is in accordance with a characteristic comprising at least one of size, mass, density, or charge.
(11) The method according to the above (5), wherein the step of isolating comprises eluting the microbe or microbe components from the substrate.
(12) The method according to the above (11), wherein the step of eluting comprises heating to a temperature of at least 70° C. with or without agitation.
(13) The method according to the above (12), wherein the heating to a temperature of at least 70° C. is performed in calcium-free water.
(14) The method according to the above (11), wherein the step of eluting comprises a pH treatment.
(15) The method according to the above (11), wherein the step of eluting comprises treatment with a chelation agent.
(16) The method according the above (15), wherein the chelating agent comprises at least one of ethylenediaminetetraacetic acid (EDTA), calcium disodium edetate (CaNa₂EDTA), ethylene glycol-bis(3-aminoethyl ether)-N,N,N′,N′-tetraacetic acid (EGTA), 1,2-bis(o-aminophenoxy)ethane-N,N,N′,N′-tetraacetic acid (BAPTA), deferoxamine mesylate salt (DFOM).
(17) The method according to the above (5), wherein the step of isolating comprises concentrating the microbe or microbe components in the sample.
(18) The method according to the above (17), wherein the isolated volume is less than the volume of the sample.
(19) The method according to the above (2), wherein the antimicrobial mixture comprises at least one of an antibiotic mixture, an antifungal mixture, and an antiviral mixture.
(20) The method according to the above (19), wherein the antibiotic mixture comprises one or more antibiotics from at least one antibiotic class comprising Cephalosporin, Glycopeptide, Cyclic lipopeptide, Aminoglycoside, Macrolide, Oxazolidinone, Fluoroquinolones, Lincosamides, and Carbapenem.
(21) The method according to the above (19), wherein the antifungal mixture comprises one or more antifungals from at least one antifungal class comprising Polyenes, Azoles, Nucleoside Analog, Echinocandin, and Allylamine.
(22) The method according to the above (19), wherein the antiviral mixture comprises one or more antivirals from at least one antiviral class comprising CCR5 anatonists, Fusion inhibitors, Nucleoside/Nucleotide reverse transcriptase inhibitors (NRTIs), Non-nucleoside reverse transcriptase inhibitors (NNRTIs), Nucleotide reverse transcriptase inhibitors (NtRTIs), Integrase inhibitors, Protease inhibitors, DNA polymerase inhibitors, Guanosine analogs, Interferon-alpha, M2 ion channel blockers, Nucleoside inhibitors, NS5A polymerase inhibitors, NS3/4A protease inhibitors, Neuraminidase inhibitors, Nucleoside analogs, and Direct acting antivirals (DAAs).
(23) The method according to the above (19), wherein the antibiotic mixture comprises one or more of Cefepime, Vancomycin, Daptomycin, Amikacin, Erythromycin, Linezolid, Ciproflaxin, Lincomycin, and Meropenem.
(24) The method according to the above (19), wherein the antifungal mixture comprises one or more of Caspofungin or Amphotericin.
(25) The method according to the above (2), wherein the antimicrobial mixture comprises one or more of Cefepime, Vancomycin, Daptomycin, Amikacin, Erythromycin, Linezolid, Ciproflaxin, Lincomycin, Meropenem, Caspofungin and Amphotericin.
(26) The method according to the above (19), wherein the antimicrobial mixture comprises at least one antibiotic mixture at a concentration from about 0.1 ug/mL to about 100 mg/mL.
(27) The method according to the above (19), wherein the antimicrobial mixture comprises at least one antifungal mixture at a concentration from about 0.01 ug/mL to about 100 mg/mL.
(28) The method according to the above (1), wherein the conditions promoting digestion include heating the sample.
(29) The method according to the above (1), further comprising contacting the digested microbe or microbe components with a composition that is more acidic than the digested microbe or microbe components.
(30) The method according to the above (29), wherein the composition that is more acidic than the digested microbe or microbe components is present at a volume equal to or greater than the volume of the digested microbe or microbe components.
(31) The method according to the above (29), wherein the composition that is more acidic than the digested microbe or microbe components comprises trifluoroacetic acid (TFA), formic acid, or acetic acid.
(32) The method according to the above (1), wherein the target substrate is evenly sprayed with matrix solution prior to analyzing microbe or microbe components to generate a homogenous layer of crystallized matrix on top of the substrate.
(33) The method according to the above (1), further comprising analyzing the microbe or microbe components.
(34) The method according to the above (33), wherein the analyzing step is performed using a method comprising at least one of volatile organic compound method; Raman spectroscopy; FFT (Fast-Fourier Transform); Fourier-Transform Infrared Spectroscopy (FTIR); infrared spectrometry; Nuclear Magnetic Resonance (NMR) spectrometry; chromatographic method, or mass spectrometric method.
(35) The method according to the above (34), wherein the analyzing step is performed via a mass spectrometric method.
(36) The method according to the above (35), wherein the mass spectrometric method comprises at least one of electron ionization, chemical ionization, electrospray ionization, atmospheric pressure chemical ionization, and matrix-assisted laser desorption ionization (MALDI-TOF MS).
(37) The method according to the above (35), wherein the mass spectrometric method is automated.
(38) The method according to the above (1), wherein the sample comprises blood, serum, plasma, sputum, urine, joint fluid, or any other tissue or biological sample.
(39) The method according to the above (1), further comprising optionally culturing the sample prior to adding the substance.
(40) The method according to the above (5), wherein the microbe-targeting molecule comprises a microbe surface-binding domain.
(41) The method according to the above (40), wherein the microbe surface-binding domain comprises a mannose-binding lectin (MBL).
(42) The method according to the above (41), wherein the microbe surface-binding domain comprises a human mannose-binding lectin (MBL).
(43) The method according to the above (40), wherein the microbe surface-binding domain comprises a carbohydrate recognition domain (CRD) of MBL.
(44) The method according to the above (43), wherein the CRD is linked to an immunoglobulin or fragment thereof.
(45) The method according to the above (43), wherein the CRD is linked to an Fc component of human IgG1 (FcMBL).
(46) The method according to the above (6), wherein the magnetic substrate is a superparamagnetic substrate.
(47) The method according to the above (6), wherein the magnetic substrate comprises at least one of a magnetic bead, a superparamagnetic bead, or a magnetic microbead.
(48) The method according to the above (5), wherein the microbe-targeting molecule is linked to an ELISA plate.
(49) The method according to the above (1), wherein the microbe comprises a Gram-positive bacterial species, a Gram-negative bacterial species, a mycobacterium, a fungus, a parasite, a bacterial antigen, a viral antigen, a protozoan, an alga, or a virus.
(50) The method according to the above (1), wherein the microbe component comprises a component from a Gram-positive bacterial species, a Gram-negative bacterial species, a mycobacterium, a fungus, a parasite, a bacterial antigen, a viral antigen, a protozoan, an alga, or a virus.
(51) The method according to the above (1), wherein the microbe component comprises microbe-associated molecular patterns (MAMPs) and/or microbe-associated proteins.
(52) The method according to the above (1), wherein the microbe is a pathogen that affects humans.
(53) The method according to the above (1), wherein the microbe or microbe component is a pathogen or component thereof.
(54) The method according to the above (53), further comprising identifying the group of the pathogen
(55) The method according to the above (53), further comprising identifying the domain of the pathogen.
(56) The method according to the above (53), further comprising identifying the species of the pathogen.
(57) The method according to the above (53), further comprising identifying the strain of the pathogen.
(58) The method according to the above (53), further comprising identifying the antimicrobial susceptibility of the pathogen.
(59) A method of preparing a sample for detecting a microbe or microbe components present in the sample, the method comprising isolating from a sample a microbe or microbe components bound to a microbe-targeting molecule on a substrate; adding a substance to the isolated microbe or microbe components; digesting the isolated microbe or microbe components under conditions promoting digestion of the microbe or microbe components; and contacting the digested microbe or microbe components with a matrix or matrix solution on a target substrate.

### BRIEF DESCRIPTION OF THE DRAWINGS

FIG. 1 illustrates unique spectra obtained by a mass spectrometer for seven pathogens where the samples were untreated, treated with one antibiotic, and treated with an antibiotic mixture.

FIG. 2 illustrates the spectra of FIG. 1 with increased gain for visualization.

FIG. 3 illustrates unique spectra obtained by a mass spectrometer for nine pathogens where the samples were untreated, treated with one antibiotic, and treated with an antibiotic mixture.

FIG. 4 illustrates the spectra of FIG. 3 with increased gain for visualization.

FIG. 5 illustrates unique spectra obtained for a single pathogen, utilizing different sample preparation methods.

### DETAILED DESCRIPTION

### I. Definitions

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

The present invention is generally directed to infectious disease diagnostic devices and methods and methods for preparing a sample comprising microbe or microbe components for analysis. The diagnostic devices of the invention can be used for analysis of a sample, for example, in the detection and/or identification of microbes in a sample.

A method of preparing a sample for detecting a microbe or microbe component according to the present invention can comprise digesting the microbe or microbe components with a substance and optionally contacting the microbe or microbe components with a matrix or matrix solution.

In some aspects, the method can further comprise, prior to adding the substance, culturing the sample to grow the microbe or microbe components. In some aspects, the method can further comprise, prior to adding the substance, contacting the sample with an MTM bound substrate and isolating from a sample a microbe or microbe components bound to an MTM on a substrate.

As used herein, “MTM” and “engineered MTM” refers to any of the molecules described herein (or described in patents or patent application incorporated by reference) that can bind to a microbe or microbe component. Unless the context indicates otherwise, the term “MTM” is used to describe all MTMs of the invention, both naturally-occurring and engineered forms of these constructs. The terms “microbe-targeting molecule” and “microbe-binding molecule” are used interchangeably herein.

A characteristic of the MTMs used in the devices, systems and methods of the invention is that these constructs contact and bind microbes and microbial components in a sample based on the identity of the MAMP produced by the microbe, rather than the identity of microbe itself. While some MAMPs are produced by only a single species of microbe, other MAMPs are shared across species. Thus, while some MTMs of the invention bind to only MAMPs of a particular species of microbe, other MTMs of the invention can bind to MAMPs produced by all members of a particular class, order, family, genus or sub-genus of microbe.

### MAMPs

As indicated above, each of the MTMs bind to at least one microbe-associated molecular pattern (MAMP). Some MTMs bind at least two, at least three, at least four, at least five, or more than five MAMPs.

As used herein and throughout the specification, the term “microbe-associated molecular patterns” or “MAMPs” refers to molecules, components or motifs associated with or secreted or released by microbes or groups of microbes (whole and/or lysed and/or disrupted) that are generally recognized by corresponding pattern recognition receptors (PRRs) of the MTM microbe-binding domains defined herein. In some aspects, the MAMPs encompass molecules associated with cellular components released during cell damage or lysis. Examples of MAMPs include, but are not limited to, microbial carbohydrates (e.g., lipopolysaccharide or LPS, mannose), endotoxins, microbial nucleic acids (e.g., bacterial, fungal or viral DNA or RNA; e.g., nucleic acids comprising a CpG site), microbial peptides (e.g., flagellin), peptidoglycans, lipoteichoic acids, N-formylmethionine, lipoproteins, lipids, phospholipids or their precursors (e.g., phosphochloline), and fungal glucans.

In some aspects, microbe components comprise cell wall or membrane components known as pathogen-associated molecular patterns (PAMPs) including lipopolysaccharide (LPS) endotoxin, lipoteichoic acid, and attached or released outer membrane vesicles. In some aspects, a microbe comprises a host cell membrane and a pathogen component or a PAMP.

In some aspects, microbe components comprise damage-associated molecular patterns (DAMPs), also known as danger-associated molecular patterns, danger signals, and alarmin. These biomolecules can initiate and sustain a non-infectious inflammatory response in a subject, in contrast to PAMPs which initiate and sustain an infectious pathogen-induced inflammatory response. Upon release from damaged or dying cells, DAMPs activate the innate immune system through binding to pattern recognition receptors (PRRs). DAMPs are recognized by immune receptors, such as toll-like receptors (TLRs) and NOD-like receptor family, pyrin domain containing 3 (NLRP3), expressed by sentinel cells of the immune system. DAMPs include portions of nuclear and cytosolic proteins, ECM (extracellular matrix), mitochondria, granules, ER (endoplasmic reticulum), and plasma membrane.

In some aspects, MAMPs include carbohydrate recognition domain (CRD)-binding motifs. As used herein, the term “carbohydrate recognition domain (CRD)-binding motifs” refers to molecules or motifs that are bound by a molecule or composition comprising a CRD (i.e. CRDs recognize and bind to CRD-binding motifs). As used herein, the term “carbohydrate recognition domain” or “CRD” refers to one or more regions, at least a portion of which, can bind to carbohydrates on a surface of microbes or pathogens. In some aspects, the CRD can be derived from a lectin, as described herein. In some aspects, the CRD can be derived from a mannan-binding lectin (MBL). Accordingly, in some aspects, MAMPs are molecules, components or motifs associated with microbes or groups of microbes that are recognized by lectin-based MTMs (collectin-based MTMs) described herein that have a CRD domain. In one embodiment, MAMPs are molecules, components, or motifs associated with microbes or groups of microbes that are recognized by mannan-binding lectin (MBL).

In some aspects, MAMPs are molecules, components or motifs associated with microbes or groups of microbes that are recognized by a C-reactive protein (CRP)-based MTMs (collectin-based MTMs).

For clarity, MAMPs as used herein includes microbe components such as MAMPs, PAMPs and DAMPs as defined above.

When necessary, and unless otherwise detectable without pre-treatment, MAMPs can be exposed, released or generated from microbes in a sample by various sample pretreatment methods. In some aspects, the MAMPs can be exposed, released or generated by lysing or killing at least a portion of the microbes in the sample. Without limitations, any means known or available to the practitioner for lysing or killing microbe cells can be used. Exemplary methods for lysing or killing the cells include, but are not limited to, physical, mechanical, chemical, radiation, biological, and the like. Accordingly, pre-treatment for lysing and/or killing the microbe cells can include application of one or more of ultrasound waves, vortexing, centrifugation, vibration, magnetic field, radiation (e.g., light, UV, Vis, IR, X-ray, and the like), change in temperature, flash-freezing, change in ionic strength, change in pH, incubation with chemicals (e.g. antimicrobial agents), enzymatic degradation, and the like.

### Microbes

As used herein, the term “microbe”, and the plural “microbes”, generally refers to microorganism(s), including bacteria, virus, fungi, parasites, protozoan, archaea, protists, e.g., algae, and a combination thereof. The term “microbe” encompasses both live and dead microbes. The term “microbe” also includes pathogenic microbes or pathogens, e.g., bacteria causing diseases such as sepsis, plague, tuberculosis and anthrax; protozoa causing diseases such as malaria, sleeping sickness and toxoplasmosis; and fungi causing diseases such as ringworm, candidiasis or histoplasmosis.

In some aspects, the microbe is a human pathogen, in other words a microbe that causes at least one disease in a human.

In some aspects, the microbe is a Gram-positive bacterial species, a Gram-negative bacterial species, a mycobacterium, a fungus, a parasite, protozoa, or a virus. In some aspects, the Gram-positive bacterial species comprises bacteria from the class Bacilli. In some aspects, the Gram-negative bacterial species comprises bacteria from the class Gammaproteobacteria. In some aspects, the mycobacterium comprises bacteria from the class Actinobacteria. In some aspects, the fungus comprises fungus from the class Saccharomycetes.

In some aspects, the microbe is Staphylococcus aureus, Streptococcus pyogenes, Klebsiella pneumoniae, Pseudomonas aeruginosa, Mycobacterium tuberculosis, Candida albicans, or Escherichia coli. In some aspects, the microbe is S. aureus strain 3518, S. pyogenes strain 011014, K. pneumoniae strain 631, E. coli strain 41949, P. aeruginosa strain 41504, C. albicans strain 1311, or M. tuberculosis strain H37Rv.

In some aspects, the microbe is Bartonella henselae, Borrelia burgdorferi, Campylobacter jejuni, Campylobacterfetus, Chlamydia trachomatis, Chlamydia pneumoniae, Chylamydia psittaci, Simkania negevensis, Escherichia coli (e.g., O157:H7 and K88), Ehrlichia chafeensis, Clostridium botulinum, Clostridium perfringens, Clostridium tetani, Enterococcus faecalis, Haemophilius influenzae, Haemophilius ducreyi, Coccidioides immitis, Bordetella pertussis, Coxiella burnetii, Ureaplasma urealyticum, Mycoplasma genitalium, Trichomatis vaginalis, Helicobacter pylori, Helicobacter hepaticus, Legionella pneumophila, Mycobacterium tuberculosis, Mycobacterium bovis, Mycobacterium africanum, Mycobacterium leprae, Mycobacterium asiaticum, Mycobacterium avium, Mycobacterium celatum, Mycobacterium celonae, Mycobacterium fortuitum, Mycobacterium genavense, Mycobacterium haemophilum, Mycobacterium intracellulare, Mycobacterium kansasii, Mycobacterium malmoense, Mycobacterium marinum, Mycobacterium scrofulaceum, Mycobacterium simiae, Mycobacterium szulgai, Mycobacterium ulcerans, Mycobacterium xenopi, Corynebacterium diptheriae, Rhodococcus equi, Rickettsia aeschlimannii, Rickettsia africae, Rickettsia conorii, Arcanobacterium haemolyticum, Bacillus anthracia, Bacillus cereus, Lysteria monocytogenes, Yersinia pestis, Yersinia enterocolitica, Shigella dysenteriae, Neisseria meningitides, Neisseria gonorrhoeae, Streptococcus bovis, Streptococcus hemolyticus, Streptococcus mutans, Streptococcus pyogenes, Streptococcus pneumoniae, Staphylococcus aureus, Staphylococcus epidermidis, Staphylococcus pneumoniae, Staphylococcus saprophyticus, Vibrio cholerae, Vibrio parahaemolyticus, Salmonella typhi, Salmonella paratyphi, Salmonella enteritidis, Treponema pallidum, Human rhinovirus, Human coronavirus such as SARS-CoV-2, Dengue virus, Filoviruses (e.g., Marburg and Ebola viruses), Hantavirus, Rift Valley virus, Hepatitis B, C, and E, Human Immunodeficiency Virus (e.g., HIV-1, HIV-2), HHV-8, Human papillomavirus, Herpes virus (e.g., HV-I and HV-II), Human T-cell lymphotrophic viruses (e.g., HTLV-I and HTLV-II), Bovine leukemia virus, Influenza virus, Guanarito virus, Lassa virus, Measles virus, Rubella virus, Mumps virus, Chickenpox (Varicella virus), Monkey pox, Epstein Bahr virus, Norwalk (and Norwalk-like) viruses, Rotavirus, Parvovirus B19, Hantaan virus, Sin Nombre virus, Venezuelan equine encephalitis, Sabia virus, West Nile virus, Yellow Fever virus, causative agents of transmissible spongiform encephalopathies, Creutzfeldt-Jakob disease agent, variant Creutzfeldt-Jakob disease agent, Candida, Cryptcooccus, Cryptosporidium, Giardia lamblia, Microsporidia, Plasmodium vivax, Pneumocystis carinii, Toxoplasma gondii, Trichophyton mentagrophytes, Enterocytozoon bieneusi, Cyclospora cayetanensis, Encephalitozoon hellem, or Encephalitozoon cuniculi, among other viruses, bacteria, archaea, protozoa, and fungi. In yet other aspects, the microbe is a bioterror agent (e.g., B. anthracis, and smallpox).

As used herein, “microbe component” and “microbial component” refer to any part of a microbe such as cell wall components, cell membrane components, cell envelope components, cytosolic components, intracellular components, nucleic acid (DNA or RNA), or organelles in the case of eukaryotic microbes. In some aspects, the microbial component comprises a component from a Gram-positive bacterial species, a Gram-negative bacterial species, a mycobacterium, a fungus, a parasite, a virus, or any microbe described herein or known in the art.

### Sample

A sample can include but is not limited to, a patient sample, an animal or animal model sample, an agricultural sample, a food and beverage sample, an environmental sample, a pharmaceutical sample, a biological sample, and a non-biological sample. A biological sample can include but is not limited to, cells, tissue, peripheral blood, and a bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The biological sample can be collected from any source, including, e.g., human or animal suspected of being infected or contaminated by a microbe(s). Biological fluids can include a bodily fluid and may be collected in any clinically acceptable manner. Biological fluids can include, but are not limited to, mucous, phlegm, saliva, sputum, blood, plasma, serum, serum derivatives, bile, sweat, amniotic fluid, menstrual fluid, mammary fluid, peritoneal fluid, interstitial fluid, urine, semen, synovial fluid, interocular fluid, a joint fluid, an articular fluid, and cerebrospinal fluid (CSF). A fluid may also be a fine needle aspirate or biopsied tissue. Blood fluids can be obtained by standard phlebotomy procedures and may be separated into components such as plasma for analysis. Centrifugation can be used to separate out fluid components to obtain plasma, buffy coat, erythrocytes, cells, pathogens and other components.

In some aspects, the sample, such as a fluid, may be purified before introduction to a device or a system of the invention. For example, filtration or centrifugation to remove particulates and chemical interference may be used. Various filtration media for removal of particles includes filter paper, such as cellulose and membrane filters, such as regenerated cellulose, cellulose acetate, nylon, PTFE, polypropylene, polyester, polyethersulfone, polyarylethersulfone, polycarbonate, and polyvinylpyrolidone.

Environmental samples include, but are not limited to, air samples, liquid and fluid samples, and dry samples. Suitable air samples include, but are not limited to, an aerosol, an atmospheric sample, and a ventilator discharge. Suitable dry samples include, but are not limited to, soil. Environmental fluids include, for example, saturated soil water, groundwater, surface water, unsaturated soil water; and fluids from industrialized processes such as waste water. Agricultural fluids can include, for example, crop fluids, such as grain and forage products, such as soybeans, wheat, and corn.

Pharmaceutical samples include, but are not limited to, drug material samples and therapeutic fluid samples, for example, for quality control or detection of endotoxins. Suitable therapeutic fluids include, but are not limited to, a dialysis fluid.

### MTMs

In some aspects, the devices may comprise MTMs that bind to one or more MAMPs. The diagnostic devices of the invention can be used for sample analysis, for example, in the detection and/or identification of microbes and microbe components in a sample.

MTMs distinguish and bind microbes and microbial components from a sample based on the identity of the MAMP produced by the microbe, rather than the identity of microbe itself. While some MAMPs are produced by only a single species of microbe, other MAMPs are shared across species. Thus, while some MTMs of the invention bind to only MAMPs of a particular species of microbe, other MTMs of the invention can bind to MAMPs produced by all members of a particular class, order, family, genus or sub-genus of microbe.

As will be apparent, while the “MTMs” of the invention include naturally-occurring molecules and proteins, the “engineered MTMs” of the invention are those have been manipulated in some manner by the hand of man. As used herein and throughout the specification, the term “engineered MTM” includes any non-naturally-occurring MTM. Engineered MTMs of the invention retain the binding specificity to a MAMP of the wild-type (i.e. naturally-occurring) molecule on which the engineered MTM is based.

The MTMs of the invention are defined based on their binding activity, therefore both naturally-occurring and engineered MTMs will comprise at least one microbe-binding domain, i.e. a domain that recognizes and binds to one or more MAMPs (including, at least two, at least three, at least four, at least five, or more) as described herein. A microbe-binding domain can be a naturally-occurring or a synthetic molecule. In some aspects, a microbe-binding domain can be a recombinant molecule.

Acceptable microbe-binding domains for use in the MTMs of the invention are limited only in their ability to recognize and bind at least one MAMP. In some aspects, the microbe-binding domain may comprise some or all of a peptide; polypeptide; protein; peptidomimetic; antibody; antibody fragment; antigen-binding fragment of an antibody; carbohydrate-binding protein; lectin; glycoprotein; glycoprotein-binding molecule; amino acid; carbohydrate (including mono-; di-; tri- and poly-saccharides); lipid; steroid; hormone; lipid-binding molecule; cofactor; nucleoside; nucleotide; nucleic acid; DNA; RNA; analogues and derivatives of nucleic acids; peptidoglycan; lipopolysaccharide; small molecule; endotoxin; bacterial lipopolysaccharide; and any combination thereof.

In particular aspects, the microbe-binding domain can be a microbe-binding domain of a lectin. An exemplary lectin is mannan binding lectin (MBL) or other mannan binding molecules. Non-limiting examples of acceptable microbe-binding domains also include microbe-binding domains from toll-like receptors, nucleotide oligomerization domain-containing (NOD) proteins, complement receptors, collectins, ficolins, pentraxins such as serum amyloid and C-reactive protein, lipid transferases, peptidoglycan recognition proteins (PGRs), and any combinations thereof. In some aspects, microbe-binding domains can be microbe-binding molecules described in the International Patent Application No. WO 2013/012924, the contents of which are incorporated by reference in their entirety.

The MTMs of the invention will typically have one or more domains in addition to a microbe-binding domain. Such domains include, but are not limited to, an oligomerization domain, a signal domain, an anchor domain, a collagen-like domain, a fibrinogen-like domain, an immunoglobulin domain, and an immunoglobulin-like domain.

Engineered MTMs of the invention include, but are not limited to, MTMs identical to a naturally-occurring MTM but having at least one amino acid change in comparison to the wild-type molecule on which they are based. Such “sequence-variant engineered MTMs” have at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99 sequence identity, though in all cases less than 100% sequence identity, to the wild-type molecule on which they are based. The changes may be any combination of additions, insertions, deletions and substitutions, where the altered amino acids may be naturally-occurring or non-naturally-occurring amino acids, and conservative or non-conservative changes.

Engineered MTMs of the invention also include, but are not limited to, MTMs that comprise domains from two or more different MTMs, i.e. fusion proteins. Such “domain-variant engineered MTMs” have domains from 2, 3, 4, 5 or more different proteins. For example, MTMs can be a fusion protein comprising a microbe-binding domain and an oligomerization domain, or a fusion protein comprising a microbe-binding domain and a signal domain, or a fusion protein comprising a microbe-binding domain, an oligomerization domain, and signal domain, to name a few examples. In each case, the domains within a domain-variant engineered MTM are from at least two different proteins. Other examples of such MTMs include fusion proteins comprising at least the microbe-binding domain of a lectin and at least a part of a second protein or peptide, e.g., but not limited, to an Fc portion of an immunoglobulin.

Engineered MTMs of the invention further include, but are not limited to, MTMs that comprise domains from two or more different MTMs, wherein at least one of the domains is a sequence variant of the wild-type domain upon which it is based, i.e. having at least one amino acid change in comparison to the wild-type molecule on which it is based. These “sequence- and domain-variant engineered MTMs” have domains from 2, 3, 4, 5 or more different proteins, and at least one of the domains has at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99 sequence identity, though in all cases less than 100% sequence identity, to the wild-type domain on which it is based. The changes may be any combination of additions, insertions, deletions and substitutions, where the altered amino acids may be naturally-occurring or non-naturally-occurring amino acids, and conservative or non-conservative changes.

As non-limiting examples of the MTMs of the invention, three broad categories of suitable MTMs are defined in the following paragraphs, namely: (i) collectin-based MTMs, (ii) ficolin-based MTMs, and (iii) toll-like receptor-based MTMs. It should be understood that these three categories are not the only categories of MTMs encompassed by the invention.

### Collectin-Based MTMs

The MTMs of the invention include collectin-based MTMs. These MTMs comprise at least one microbe-binding domain of a collectin, such as the lectin carbohydrate-recognition domain (CRD).

Collectins (collagen-containing C-type lectins) are a family of collagenous calcium-dependent lectins that function in defense, thus playing an important role in the innate immune system. They are soluble molecules comprising pattern recognition receptors (PRRs) within the microbe-binding domain that recognize and bind to particular oligosaccharide structures or lipids displayed on the surface of microbes, i.e. MAMPs of oligosaccharide origin. Upon binding of collectins to a microbe, clearance of the microbe is achieved via aggregation, complement activation, opsonization, and activation of phagocytosis.

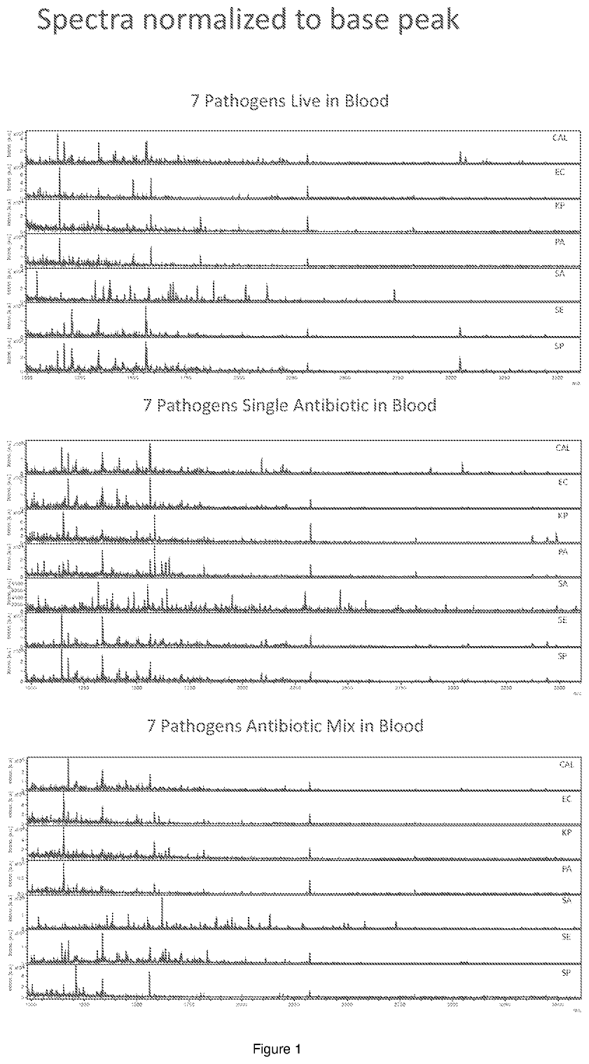

Members of the family have a common structure, characterized by four parts or domains arranged in the following N- to C-terminal arrangement: (i) a cysteine-rich domain, (ii) a collagen-like domain, (iii) a coiled-coil neck domain, and (iv) a microbe-binding domain which includes a C-type lectin domain, also termed the carbohydrate recognition domain (CRD). The functional form of the molecule is a trimer made up of three identical chains. MAMP recognition is mediated by the CRD in presence of calcium. See FIG. 1.

There are currently nine recognized members of the family: (i) mannose-binding lectin (MBL; mannan-binding lectin; e.g. SEQ ID NO:1), (ii) surfactant protein A (SP-A), (iii) surfactant protein D (SP-D), (iv) collectin liver 1 (CL-L1), (v) collectin placenta 1 (CL-P1), (vi) conglutinin collectin of 43 kDa (CL-43), (vii) collectin of 46 kDa (CL-46), (viii) collectin kidney 1 (CL-K1), and (ix) conglutinin. Each of these proteins is an MTM of the invention.

The MTMs of the invention also include other collectin-based molecules that bind to one or more MAMPs, e.g. those MTMs comprising at least a portion (e.g. domain) of a lectin-based molecule in the case of an engineered MTM. As used herein, the term “collectin-based molecule” refers to a molecule comprising a microbe-binding domain derived from a collectin, such as a lectin. The term “lectin” as used herein refers to any molecule including proteins, natural or genetically modified (e.g., recombinant), that interacts specifically with saccharides (e.g., carbohydrates). The term “lectin” as used herein can also refer to lectins derived from any species, including, but not limited to, plants, animals (e.g. mammals, such as human), insects and microorganisms, having a desired carbohydrate binding specificity. Examples of plant lectins include, but are not limited to, the Leguminosae lectin family, such as ConA, soybean agglutinin, peanut lectin, lentil lectin, and Galanthus nivalis agglutinin (GNA) from the Galanthus (snowdrop) plant. Other examples of plant lectins are the Gramineae and Solanaceae families of lectins. Examples of animal lectins include, but are not limited to, any known lectin of the major groups S-type lectins, C-type lectins, P-type lectins, and I-type lectins, and galectins. In some aspects, the carbohydrate recognition domain can be derived from a C-type lectin, or a fragment thereof. C-type lectin can include any carbohydrate-binding protein that requires calcium for binding (e.g., MBL). In some aspects, the C-type lectin can include, but are not limited to, collectin, DC-SIGN, and fragments thereof. Without wishing to be bound by theory, DC-SIGN can generally bind various microbes by recognizing high-mannose-containing glycoproteins on their envelopes and/or function as a receptor for several viruses such as HIV and Hepatitis C.

Collectin-based engineered MTMs of the invention are MTMs that comprise at least a microbe-binding domain of a collectin. These MTMs may also include one or more of the other domains of a collectin, e.g. a cysteine-rich domain, a collagen-like domain, and/or a coiled-coil neck domain, as well as one or more domains not typically found in a collectin, such as an oligomerization domain, a signal domain, an anchor domain, a collagen-like domain, a fibrinogen-like domain, an immunoglobulin domain, and/or an immunoglobulin-like domain. When a collectin-based engineered MTM has each of the domains of a wild-type collectin, the MTM will be a sequence-variant engineered MTM as defined above. When a collectin-based engineered MTM has fewer that all of the domains of a wild-type collectin, the MTM will be a domain-variant engineered MTM or a sequence- and domain-variant engineered MTM as defined above.

Collectin-based engineered MTMs comprise a microbe-binding domain derived from at least one carbohydrate-binding protein selected from the group consisting of: MBL; SP-A; SP-D; CL-L1, CL-P1; CL-34; CL-46; CL-K1, conglutinin; maltose-binding protein; arabinose-binding protein; glucose-binding protein; Galanthus nivalis agglutinin; peanut lectin; lentil lectin; DC-SIGN; and C-reactive protein; and any combinations thereof.

In some aspects, the MTMs and engineered MTMs of the invention comprise the microbe-binding domain of a mannose-binding lectin (MBL). In some aspects, the microbe-binding domain comprises a human mannose-binding lectin (MBL; SEQ ID NO: 1). In some aspects, the microbe-binding domain comprises a MBL of a primate, mouse, rat, hamster, rabbit, or any other species as described herein. In some aspects, the microbe-binding domain comprises a portion of a human MBL (see e.g., SEQ ID NOs: 2-3). In some aspects, the microbe-binding domain comprises a plant MBL. In some aspects, the microbe-binding domain comprises a carbohydrate recognition domain (CRD) of MBL (see e.g., SEQ ID NO: 4).

MBL full length (SEQ ID NO: 1):
MSLFPSLPLL LLSMVAASYS ETVTCEDAQK TCPAVIACSS PGINGFPGKD
GRDGTKGEKG EPGQGLRGLQ GPPGKLGPPG NPGPSGSPGP KGQKGDPGKS
PDGDSSLAAS ERKALQTEMA RIKKWLTFSL GKQVGNKFFL TNGEIMTFEK
VKALCVKFQA SVATPRNAAE NGAIQNLIKE EAFLGITDEK TEGQFVDLTG
NRLTYTNWNE GEPNNAGSDE DCVLLLKNGQ WNDVPCSTSH LAVCEFPI
MBL without the signal sequence (SEQ ID NO: 2):
ETVTCEDAQK TCPAVIACSS PGINGFPGKD GRDGTKGEKG EPGQGLRGLQ
GPPGKLGPPG NPGPSGSPGP KGQKGDPGKS PDGDSSLAAS ERKALQTEMA
RIKKWLTFSL GKQVGNKFFL TNGEIMTFEK VKALCVKFQA SVATPRNAAE
NGAIQNLIKE EAFLGITDEK TEGQFVDLTG NRLTYTNWNE GEPNNAGSDE
DCVLLLKNGQ WNDVPCSTSH LAVCEFPI
Truncated MBL (SEQ ID NO: 3):
AASERKALQT EMARIKKWLT FSLGKQVGNK FFLTNGEIMT FEKVKALCVK
FQASVATPRN AAENGAIQNL IKEEAFLGIT DEKTEGQFVD LTGNRLTYTN
WNEGEPNNAG SDEDCVLLLK NGQWNDVPCS TSHLAVCEFP I
Carbohydrate recognition domain (CRD) of MBL (SEQ ID NO: 4):
VGNKFFLTNG EIMTFEKVKA LCVKFQASVA TPRNAAENGA IQNLIKEEAF
LGITDEKTEG QFVDLTGNRL TYTNWNEGEP NNAGSDEDCV LLLKNGQWND
VPCSTSHLAV CEFPI

Alternatively, or in addition, the MTMs and engineered MTMs of the invention comprise the coiled-coil neck domain and a microbe-binding domain of a MBL (see, e.g. SEQ ID NO:5).

Neck + Carbohydrate recognition domain of MBL (SEQ ID NO: 5):
PDGDSSLAAS ERKALQTEMA RIKKWLTFSL GKQVGNKFFL TNGEIMTFEK
VKALCVKFQA SVATPRNAAE NGAIQNLIKE EAELGITDEK TEGQFVDLTG
NRLTYTNWNE GEPNNAGSDE DCVLLLKNGQ WNDVPCSTSH LAVCEFPI

Suitable collectin-based, domain-variant engineered MTMs of the invention include recombinant lectins such as FcMBL. FcMBL is a fusion protein comprising a carbohydrate recognition domain (CRD) of MBL and a portion of immunoglobulin. In some aspects, the FcMBL further comprises a neck region of MBL. In some aspects, the N-terminus of FcMBL can comprise an oligopeptide anchor domain adapted to bind a solid substrate and orient the CRD of MBL away from the solid substrate surface. See SEQ ID NOs: 6-8 for examples of FcMBLs of the invention. Various genetically engineered versions of MBL (e.g., FcMBL) are described in PCT application publications WO 2011/090954 and WO 2013/012924, as well as U.S. Pat. Nos. 9,150,631 and 9,593,160, the contents of each of which are incorporated herein by reference in their entireties. Lectins and other mannan binding molecules are also described in, for example, U.S. Pat. Nos. 9,150,631 and 9,632,085, and PCT application publications WO 2011/090954, WO 2013/012924, and WO 2013/130875, the contents of all of which are incorporated herein by reference in their entireties.

Amino acid sequences for suitable engineered MTMs of the invention include, but are not limited to:

FcMBL.81 (SEQ ID NO: 6):
EPKSSDKTHT CPPCPAPELL GGPSVFLFPP KPKDTLMISR TPEVTCVVVD
VSHEDPEVKF NWYVDGVEVH NAKTKPREEQ YNSTYRWSV LTVLHQDWLN
GKEYKCKVSN KALPAPIEKT ISKAKGQPRE PQVYTLPPSR DELTKNQVSL
TCLVKGFYPS DIAVEWESNG QPENNYKTTP PVLDSDGSFF LYSKLTVDKS
RWQQGNVFSC SVMHEALHNH YTQKSLSLSP GAPDGDSSLA ASERKALQTE
MARIKKWLTF SLGKQVGNKF FLTNGEINIT FEKVKALCVK FQASVATPRNA
AENGAIQNLI KEEAFLGITD EKTEGQFVDL TGNRLTYTNW NEGEPNNAGS
DEDCVLLLKN GQWNDVPCST SHLAVCEFPI
AKT-FcMBL (SEQ ID NO: 7):
AKTEPKSSDK THTCPPCPAP ELLGGPSVFL FPPKPKDTLM ISRTPEVTCV
VVDVSHEDPE VKFNWYVDGV EVHNAKTKPR EEQYNSTYRV VSVLTVLHQD
WLNGKEYKCK VSNKALPAPI EKTISKAKGQ PREPQVYTLP PSRDELTKNQ
VSLTCLVKGF YPSDIAVEWE SNGQPENNYK TTPPVLDSDG SFFLYSKLTV
DKSRWQQGNV FSCSVMHEAL HNHYTQKSLS LSPGAPDGDS SLAASERKAL
QTEMARIKKW LTFSLGKQVG NKFFLTNGEI MTFEKVKALC VKFQASVATP
RNAAENGAIQ NLIKEEAFLG ITDEKTEGQF VDLTGNRLTY TNWNEGEPNN
AGSDEDCVLL LKNGQWNDVP CSTSHLAVCE EPI
FcMBL.111 (SEQ ID NO: 8):
EPKSSDKTHT CPPCPAPELL GGPSVFLFPP KPKDTLMISR TPEVTCVVVD
VSHEDPEVKF NWYVDGVEVH NAKTKPREEQ YNSTYRVVSV LTVLHQDWLN
GKEYKCKVSN KALPAPIEKT ISKAKGQPRE PQVYTLPPSR DELTKNQVSL
TCLVKGFYPS DIAVEWESNG QPENNYKTTP PVLDSDGSFF LYSKLTVDKS
RWQQGNVFSC SVMHEALHNH YTQKSLSLSP GATSKQVGNK FFLTNGEEVI
TFEKVKALCV KFQASVATPR NAAENGAIQN LIKEEAFLGI TDEKTEGQFV
DLTGNRLTYT NWNEGEPNNA GSDEDCVLLL KNGQWNDVPC STSHLAVCEF PI

In some aspects, the engineered MTMs of the invention comprise an amino acid sequence selected from SEQ ID NO:1-SEQ ID NO:8, or an amino acid sequence that is at least 80, 81, 82, 83, 84, 85, 86, 87, 88, 89, 90, 91, 92, 93, 94, 95, 96, 97, 98, or 99% identical to any one of SEQ ID NO:1-SEQ ID NO:8, but less than 100% identical, and that retains the microbe-binding activity of the wild-type protein.

In some aspects where the immunoglobulin domain comprises a Fc region or a fragment thereof, the Fc region or a fragment thereof can comprise at least one mutation, e.g., to modify the performance of the engineered MTMs. For example, in some aspects, a half-life of the engineered MTMs comprising an Fc region described herein can be increased, e.g., by mutating an amino acid lysine (K) at the residue 232 of SEQ ID NO: 9 to alanine (A). Other mutations, e.g., located at the interface between the CH2 and CH3 domains shown in Hinton et al (2004) J Biol Chem. 279:6213-6216 and Vaccaro C. et al. (2005) Nat Biotechnol. 23: 1283-1288, can be also used to increase the half-life of the IgG1 and thus the engineered MTMs.

SEQ ID NO: 9:
EPKSSDKTHT CPPCPAPELL GGPSVFLFPP KPKDTLMISR TPEVTCVVVD
VSHEDPEVKF NWYVDGVEVH NAKTKPREEQ YNSTYRVVSV LTVLHQDWLN
GKEYKCKVSN KALPAPIEKT ISKAKGQPRE PQVYTLPPSR DELTKNQVSL
TCLVKGFYPS DIAVEWESNG QPENNYKTTP PVLDSDGSFF LYSKLTVDKS
RWQQGNVFSC SVMHEALHNH YTQKSLSLSP GA

The full-length amino acid sequence of the carbohydrate recognition domain (CRD) of MBL is shown in SEQ ID NO: 4. The microbe-binding domain comprising such a CDR of an engineered MTM described herein can have an amino acid sequence of about 10 to about 300 amino acid residues, or about 50 to about 160 amino acid residues. In some aspects, the microbe-binding domain can have an amino acid sequence of at least about 5, at least about 10, at least about 15, at least about 20, at least about 30, at least about 40, at least about 50, at least about 60, at least about 70, at least about 80, at least about 90, at least about 100, at least about 150 amino acid residues or more. Accordingly, in some aspects, the carbohydrate recognition domain of the engineered MTM molecule can comprise SEQ ID NO: 4. In some aspects, the carbohydrate recognition domain of the engineered MTM molecule can comprise a fragment of SEQ ID NO: 4. Exemplary amino acid sequences of such fragments include, but are not limited to, ND; EZN (where Z is any amino acid, e.g., P); NEGEPNNAGS (SEQ ID NO: 10) or a fragment thereof comprising EPN; GSDEDCVLL or a fragment thereof comprising E, and LLLKNGQWNDVPCST (SEQ ID NO: 11) or a fragment thereof comprising ND. Modifications to such CRD fragments, e.g., by conservative substitution, are also within the scope described herein. In some aspects, the MBL or a fragment thereof used in the microbe-binding domain of the engineered MTMs described herein can be a wild-type molecule or a recombinant molecule.

The exemplary sequences provided herein for the carbohydrate recognition domain of the engineered MTMs are not to be construed as limiting. For example, while the exemplary sequences provided herein are derived from a human, amino acid sequences of the same carbohydrate recognition domain in other species such as mice, rats, porcine, bovine, feline, and canine are known in the art and within the scope described herein.

### Ficolin-Based MTMs

Ficolins are a family of lectins that activate the lectin pathway of complement activation upon binding to a pathogen. Ficolins are soluble molecules comprising pattern recognition receptors (PRRs) within a microbe-binding domain that recognize and selectively bind acetylated compounds, typically N-acetylglucosamine (GlcNAc), produced by pathogens. The lectin pathway is activated by binding of a ficolin to an acetylated compound on the pathogen surface, which activates the serine proteases MASP-1 and MASP-2, which then cleave C4 into C4a and C4b, and cleave C2 into C2a and C2b. C4b and C2b then bind together to form C3-convertase of the classical pathway, leading to the eventual lysis of the target cell via the remainder of the steps in the classical pathway.

Members of the family have a common structure, characterized by three parts or domains arranged in the following N- to C-terminal arrangement: (i) a short N-terminal domain, (ii) a collagen-like domain, and (iii) a fibrinogen-like domain that makes up the microbe-binding domain. The functional form of the molecule is a trimer made up of three identical chains. See FIG. 1.

There are currently three recognized members of the family: ficolin 1 (M-ficolin), ficolin 2 (L-ficolin), and ficolin 3 (H-ficolin). Each of these proteins is an MTM of the invention. The amino acid sequences of the human forms of the proteins are provided in the following paragraphs, with the fibrinogen-like domain underlined:

Ficolin 1 precursor - SEQ ID NO: 12 - NP_001994.2 - Homo sapiens
MELSGATMAR GLAVLLVLFL HIKNLPAQAA DTCPEVKVVG LEGSDKLTIL RGCPGLPGAP
GPKGEAGVIG ERGERGLPGA PGKAGPVGPK GDRGEKGMRG EKGDAGQSQS CATGPRNCKD
LLDRGYFLSG WHTIYLPDCR PLTVLCDMDT DGGGWTVFQR RMDGSVDFYR DWAAYKQGFG
SQLGEFWLGN DNIHALTAQG SSELRVDLVD FEGNHQFAKY KSFKVADEAE KYKLVLGAFV
GGSAGNSLTG HNNNFFSTKD QDNDVSSSNC AEKFQGAWWY ADCHASNLNG LYLMGPHESY
ANGINWSAAK GYKYSYKVSE MKVRPA
Ficolin 2 isoform a precursor - SEQ ID NO: 13 - NP_004099.2 -
Homo sapiens
MELDRAVGVL GAATLLLSFL GMAWALQAAD TCPEVKMVGL EGSDKLTILR GCPGLPGAPG
PKGEAGTNGK RGERGPPGPP GKAGPPGPNG APGEPQPCLT GPRTCKDLLD RGHFLSGWHT
IYLPDCRPLT VLCDMDTDGG GWTVFQRRVD GSVDFYRDWA TYKQGFGSRL GEFWLGNDNI
HALTAQGTSE LRVDLVDFED NYQFAKYRSF KVADEAEKYN LVLGAFVEGS AGDSLTFHNN
QSFSTKDQDN DLNTGNCAVM FQGAWWYKNC HVSNLNGRYL RGTHGSFANG INWKSGKGYN
YSYKVSEMKV RPA
Ficolin 3 isoform 1 precursor - SEQ ID NO: 14 - NP_003656.2 -
Homo sapiens
MDLLWILPSL WLLLLGGPAC LKTQEHPSCP GPRELEASKV VLLPSCPGAP GSPGEKGAPG
PQGPPGPPGK MGPKGEPGDP VNLLRCQEGP RNCRELLSQG ATLSGWYHLC LPEGRALPVF
CDMDTEGGGW LVFQRRQDGS VDFFRSWSSY RAGFGNQESE FWLGNENLHQ LTLQGNWELR
VELEDFNGNR TFAHYATFRL LGEVDHYQLA LGKFSEGTAG DSLSLHSGRP FTTYDADHDS
SNSNCAVIVH GAWWYASCYR SNLNGRYAVS EAAAHKYGID WASGRGVGHP YRRVRMMLR

The MTMs of the invention also include other ficolin-based molecules that bind to one or more MAMPs (acetylated compounds for the ficolins), e.g. those MTMs comprising at least a portion (e.g. domain) of a ficolin-based molecule in the case of an engineered MTM. As used herein, the term “ficolin-based molecule” refers to a molecule comprising a microbe-binding domain derived from a ficolin. The term “ficolin” as used herein refers to any molecule including proteins, natural or genetically modified (e.g., recombinant), that interacts specifically with acetylated compounds (e.g., GlcNAc). The term “ficolin” as used herein can also refer to ficolins derived from any species, including, but not limited to, plants, animals (e.g. mammals, such as human), insects and microorganisms, having the desired binding specificity.

Ficolin-based engineered MTMs of the invention are MTMs that comprise at least a microbe-binding domain of a ficolin, e.g. the fibrinogen-like domain of a ficolin. These MTMs may also include one or more of the other domains of a ficolin, e.g. a short N-terminal domain and/or a collagen-like domain, as well as one or more domains not typically found in a ficolin, such as an oligomerization domain, a signal domain, an anchor domain, a collagen-like domain, a fibrinogen-like domain, an immunoglobulin domain, and/or an immunoglobulin-like domain. When a ficolin-based engineered MTM has each of the domains of a wild-type ficolin, the MTM will be a sequence-variant engineered MTM as defined above. When a ficolin-based engineered MTM has fewer that all of the domains of a wild-type ficolin, the MTM will be a domain-variant engineered MTM or a sequence- and domain-variant engineered MTM as defined above.

Ficolin-based engineered MTMs comprise a microbe-binding domain comprising at least one fibrinogen-like domain of a ficolin selected from the group consisting of ficolin 1, ficolin 2, and ficolin 3.

In some aspects, the MTMs and engineered MTMs of the invention comprise a microbe-binding domain comprising the fibrinogen-like domain of ficolin 1 of SEQ ID NO:12. In other aspects, the MTMs and engineered MTMs of the invention comprise a microbe-binding domain comprising the fibrinogen-like domain of ficolin 2 of SEQ ID NO: 13. In further aspects, the MTMs and engineered MTMs of a microbe-binding domain comprising the fibrinogen-like domain of ficolin 3 of SEQ ID NO:14.

In some aspects, the microbe-binding domain comprising a fibrinogen-like domain of a ficolin from a primate, mouse, rat, hamster, rabbit, or any other species as described herein.

The exemplary sequences provided herein for the ficolins are not to be construed as limiting. For example, while the exemplary sequences provided herein are derived from a human, amino acid sequences of ficolins from other species such as mice, rats, porcine, bovine, feline, and canine are known in the art and within the scope described herein.

### Toll-Like Receptor-Based MTMs

Toll-like receptors (TLRs) comprise a family of proteins that are integral to the proper functioning of the innate immune system. The proteins are type I integral membrane proteins (i.e. single-pass, membrane-spanning receptors) that are typically found on the surface of sentinel cells, such as macrophages and dendritic cells, but can also be found on the surface of other leukocytes including natural killer cells, T cells and B cells, and non-immune cells including epithelial cell, endothelial cells, and fibroblasts. After microbes have gained entry to a subject, such as a human, through the skin or mucosa, they are recognized by TLR-expressing cells, which leads to innate immune responses and the development of antigen-specific acquired immunity. TLRs thus recognize MAMPs by microbes.

Members of the family have a common structure, characterized by three parts or domains arranged in the following N- to C-terminal arrangement: (i) an N-terminal ligand-binding domain, i.e. the microbe-binding domain, (ii) a single transmembrane helix (˜20 amino acids), and (iii) a C-terminal cytoplasmic signaling domain.

The ligand-binding domain is a glycoprotein comprising 550-800 amino acid residues (depending on the identity of the TLR), constructed of tandem copies of leucine-rich repeats (LRR), which are typically 22-29 residues in length and that contains hydrophobic residues spaced at distinctive intervals. The receptors share a common structural framework in their extracellular, ligand-binding domains. The domains each adopt a horseshoe-shaped structure formed by the leucine-rich repeat motifs.

The functional form of a TLR is a dimer, with both homodimers and heterodimers being known. In the case of heterodimers, the different TLRs in the dimer may have different ligand specificities. Upon ligand binding, TLRs dimerize their ectodomains via their lateral faces, forming “m”-shaped structures. Dimerization leads to downstream signaling.

A set of endosomal TLRs comprising TLR3, TLR7, TLR8 and TLR9 recognize nucleic acids derived from viruses as well as endogenous nucleic acids in context of pathogenic events. Activation of these receptor leads to production of inflammatory cytokines as well as type I interferons (interferon type I) to help fight viral infection.

There are a number of recognized human members of the family, including TLR1, TLR2, TLR3, TLR4, TLR5, TLR6, TLR7, TLR8, TLR9, and TLR10. Each of these proteins is an MTM of the invention. The amino acid sequences of the human forms of the proteins are provided in the following paragraphs, with the extracellular domain that comprises the N-terminal ligand-bindin domain underlined:

TLR1 (toll-like receptor 1 precursor) - NCBI Reference Sequence:
NP_003254.2
(SEQ ID NO:15)
MTSIFHFAII FMLILQIRIQ LSEESEFLVD RSKNGLIHVP KDLSQKTTIL NISQNYISEL
WTSDILSLSK LRILIISHNR IQYLDISVFK FNQELEYLDL SHNKLVKISC HPTVNLKHLD
LSFNAFDALP ICKEFGNMSQ LKFLGLSTTH LEKSSVLPIA HLNISKVLLV LGETYGEKED
PEGLQDFNTE SLHIVFPTNK EFHFILDVSV KTVANLELSN IKCVLEDNKC SYFLSILAKL
QTNPKLSNLT LNNIETTWNS FIRILQLVWH TTVWYFSISN VKLQGQLDFR DFDYSGTSLK
ALSIHQVVSD VFGFPQSYIY EIFSNMNIKN FTVSGTRMVH MLCPSKISPF LHLDFSNNLL
TDTVFENCGH LTELETLILQ MNQLKELSKI AEMTTQMKSL QQLDISQNSV SYDEKKGDCS
WTKSLLSLNM SSNILTDTIF RCLPPRIKVL DLHSNKIKSI PKQVVKLEAL QELNVAFNSL
TDLPGCGSFS SLSVLIIDHN SVSHPSADFF QSCQKMRSIK AGDNPFQCTC ELGEFVKNID
QVSSEVLEGW PDSYKCDYPE SYRGTLLKDF HMSELSCNIT LLIVTIVATM LVLAVTVTSL
CSYLDLPWYL RMVCQWTQTR RRARNIPLEE LQRNLQFHAF ISYSGHDSFW VKNELLPNLE
KEGMQICLHE RNFVPGKSIV ENIITCIEKS YKSIFVLSPN FVQSEWCHYE LYFAHHNLFH
EGSNSLILIL LEPIPQYSIP SSYHKLKSLM ARRTYLEWPK EKSKRGLFWA NLRAAINIKL
TEQAKK
TLR2 (toll-like receptor 2 precursor) - NCBI Reference Sequence:
NP_001305722.1
(SEQ ID NO:16)
MPHTLWMVWV LGVIISLSKE ESSNQASLSC DRNGICKGSS GSLNSIPSGL TEAVKSLDLS
NNRITYISNS DLQRCVNLQA LVLTSNGINT IEEDSFSSLG SLEHLDLSYN YLSNLSSSWF
KPLSSLTFLN LLGNPYKTLG ETSLFSHLTK LQILRVGNMD TFTKIQRKDF AGLTFLEELE
IDASDLQSYE PKSLKSIQNV SHLILHMKQH ILLLEIFVDV TSSVECLELR DTDLDTFHFS
ELSTGETNSL IKKFTFRNVK ITDESLFQVM KLLNQISGLL ELEFDDCTLN GVGNFRASDN
DRVIDPGKVE TLTIRRLHIP RFYLFYDLST LYSLTERVKR ITVENSKVFL VPCLLSQHLK
SLEYLDLSEN LMVEEYLKNS ACEDAWPSLQ TLILRQNHLA SLEKTGETLL TLKNLTNIDI
SKNSFHSMPE TCQWPEKMKY LNLSSTRIHS VTGCIPKTLE ILDVSNNNLN LFSLNLPQLK
ELYISRNKLM TLPDASLLPM LLVLKISRNA ITTFSKEQLD SFHTLKTLEA GGNNFICSCE
FLSFTQEQQA LAKVLIDWPA NYLCDSPSHV RGQQVQDVRL SVSECHRTAL VSGMCCALFL
LILLTGVLCH RFHGLWYMKM MWAWLQAKRK PRKAPSRNIC YDAFVSYSER DAYWVENLMV
QELENFNPPF KLCLHKRDFI PGKWIIDNII DSIEKSHKTV FVLSENFVKS EWCKYELDFS
HFRLFDENND AAILILLEPI EKKAIPQRFC KLRKIMNTKT YLEWPMDEAQ REGFWVNLRA
AIKS
TLR3 (toll-like receptor 3 precursor) - NCBI Reference Sequence:
NP_003256.1
(SEQ ID NO:17)
MRQTLPCIYF WGGLLPFGML CASSTTKCTV SHEVADCSHL KLTQVPDDLP TNITVLNLTH
NQLRRLPAAN FTRYSQLTSL DVGFNTISKL EPELCQKLPM LKVLNLQHNE LSQLSDKTFA
FCTNLTELHL MSNSIQKIKN NPFVKQKNLI TLDLSHNGLS STKLGTQVQL ENLQELLLSN
NKIQALKSEE LDIFANSSLK KLELSSNQIK EFSPGCFHAI GRLFGLFLNN VQLGPSLTEK
LCLELANTSI RNLSLSNSQL STTSNTTFLG LKWTNLTMLD LSYNNLNVVG NDSFAWLPQL
EYFFLEYNNI QHLFSHSLHG LFNVRYLNLK RSFTKQSISL ASLPKIDDFS FQWLKCLEHL
NMEDNDIPGI KSNMFTGLIN LKYLSLSNSF TSLRTLTNET FVSLAHSPLH ILNLTKNKIS
KIESDAFSWL GHLEVLDLGL NEIGQELTGQ EWRGLENIFE IYLSYNKYLQ LTRNSFALVP
SLQRLMLRRV ALKNVDSSPS PFQPLRNLTI LDLSNNNIAN INDDMLEGLE KLEILDLQHN
NLARLWKHAN PGGPIYFLKG LSHLHILNLE SNGFDEIPVE VFKDLFELKI IDLGLNNLNT
LPASVFNNQV SLKSLNLQKN LITSVEKKVF GPAFRNLTEL DMRFNPFDCT CESIAWFVNW
INETHTNIPE LSSHYLCNTP PHYHGFPVRL FDTSSCKDSA PFELFFMINT SILLIFIFIV
LLIHFEGWRI SFYWNVSVHR VLGFKEIDRQ TEQFEYAAYI IHAYKDKDWV WEHFSSMEKE
DQSLKFCLEE RDFEAGVFEL EAIVNSIKRS RKIIFVITHH LLKDPLCKRF KVHHAVQQAI
EQNLDSIILV FLEEIPDYKL NHALCLRRGM FKSHCILNWP VQKERIGAFR HKLQVALGSK
NSVH
TLR4 (toll-like receptor 4 isoform D) - NCBI Reference Sequence:
NP_612567.1
(SEQ ID NO:18)
MMSASRLAGT LIPAMAFLSC VRPESWEPCV EVVPNITYQC MELNFYKIPD NLPFSTKNLD
LSFNPLRHLG SYSFFSFPEL QVLDLSRCEI QTIEDGAYQS LSHLSTLILT GNPIQSLALG
AFSGLSSLQK LVAVETNLAS LENFPIGHLK TLKELNVAHN LIQSFKLPEY FSNLTNLEHL
DLSSNKIQSI YCTDLRVLHQ MPLLNLSLDL SLNPMNFIQP GAFKEIRLHK LTLRNNFDSL
NVMKTCIQGL AGLEVHRLVL GEFRNEGNLE KFDKSALEGL CNLTIEEFRL AYLDYYLDDI
IDLFNCLTNV SSFSLVSVTI ERVKDFSYNF GWQHLELVNC KFGQFPTLKL KSLKRLTFTS
NKGGNAFSEV DLPSLEFLDL SRNGLSFKGC CSQSDFGTTS LKYLDLSFNG VITMSSNFLG
LEQLEHLDFQ HSNLKQMSEF SVFLSLRNLI YLDISHTHTR VAFNGIFNGL SSLEVLKMAG
NSFQENFLPD IFTELRNLTF LDLSQCQLEQ LSPTAFNSLS SLQVLNMSHN NFFSLDTFPY
KCLNSLQVLD YSLNHIMTSK KQELQHFPSS LAFLNLTQND FACTCEHQSF LQWIKDQRQL
LVEVERMECA TPSDKQGMPV LSLNITCQMN KTIIGVSVLS VLVVSVVAVL VYKFYFHLML
LAGCIKYGRG ENIYDAFVIY SSQDEDWVRN ELVKNLEEGV PPFQLCLHYR DFIPGVAIAA
NIIHEGFHKS RKVIVVVSQH FIQSRWCIFE YEIAQTWQFL SSRAGIIFIV LQKVEKTLLR
QQVELYRLLS RNTYLEWEDS VLGRHIFWRR LRKALLDGKS WNPEGTVGTG CNWQEATSI
TLR5 (toll-like receptor 5 precursor) - NCBI Reference Sequence:
NP_003259.2
(SEQ ID NO:19)
MGDHLDLLLG VVLMAGPVFG IPSCSFDGRI AFYRFCNLTQ VPQVLNTTER LLLSFNYIRT
VTASSFPFLE QLQLLELGSQ YTPLTIDKEA FRNLPNLRIL DLGSSKIYFL HPDAFQGLFH
LFELRLYFCG LSDAVLKDGY FRNLKALTRL DLSKNQIRSL YLHPSFGKLN SLKSIDFSSN
QIFLVCEHEL EPLQGKTLSF FSLAANSLYS RVSVDWGKCM NPFRNMVLEI LDVSGNGWTV
DITGNFSNAI SKSQAFSLIL AHHIMGAGFG FHNIKDPDQN TFAGLARSSV RHLDLSHGFV
FSLNSRVFET LKDLKVLNLA YNKINKIADE AFYGLDNLQV LNLSYNLLGE LYSSNFYGLP
KVAYIDLQKN HIAIIQDQTF KFLEKLQTLD LRDNALTTIH FIPSIPDIFL SGNKLVTLPK
INLTANLIHL SENRLENLDI LYFLLRVPHL QILILNQNRF SSCSGDQTPS ENPSLEQLFL
GENMLQLAWE TELCWDVFEG LSHLQVLYLN HNYLNSLPPG VFSHLTALRG LSLNSNRLTV
LSHNDLPANL EILDISRNQL LAPNPDVFVS LSVLDITHNK FICECELSTF INWLNHTNVT
IAGPPADIYC VYPDSFSGVS LFSLSTEGCD EEEVLKSLKF SLFIVCTVTL TLFLMTILTV
TKFRGFCFIC YKTAQRLVFK DHPQGTEPDM YKYDAYLCFS SKDFTWVQNA LLKHLDTQYS
DQNRFNLCFE ERDFVPGENR IANIQDAIWN SRKIVCLVSR HFLRDGWCLE AFSYAQGRCL
SDLNSALIMV VVGSLSQYQL MKHQSIRGFV QKQQYLRWPE DLQDVGWFLH KLSQQILKKE
KEKKKDNNIP LQTVATIS
TLR6 (toll-like receptor 6 precursor) - NCBI Reference Sequence:
NP_006059.2
(SEQ ID NO:20)
MTKDKEPIVK SFHFVCLMII IVGTRIQFSD GNEFAVDKSK RGLIHVPKDL PLKTKVLDMS
QNYIAELQVS DMSFLSELTV LRLSHNRIQL LDLSVFKFNQ DLEYLDLSHN QLQKISCHPI
VSFRHLDLSF NDFKALPICK EFGNLSQLNF LGLSAMKLQK LDLLPIAHLH LSYILLDLRN
YYIKENETES LQILNAKTLH LVFHPTSLFA IQVNISVNTL GCLQLTNIKL NDDNCQVFIK
FLSELTRGST LLNFTLNHIE TTWKCLVRVF QFLWPKPVEY LNIYNLTIIE SIREEDFTYS
KTTLKALTIE HITNQVFLFS QTALYTVFSE MNIMMLTISD TPFIHMLCPH APSTFKFLNF
TQNVFTDSIF EKCSTLVKLE TLILQKNGLK DLFKVGLMTK DMPSLEILDV SWNSLESGRH
KENCTWVESI VVLNLSSNML TDSVFRCLPP RIKVLDLHSN KIKSVPKQVV KLEALQELNV
AFNSLTDLPG CGSFSSLSVL IIDHNSVSHP SADFFQSCQK MRSIKAGDNP FQCTCELREF
VKNIDQVSSE VLEGWPDSYK CDYPESYRGS PLKDFHMSEL SCNITLLIVT IGATMLVLAV
TVTSLCIYLD LPWYLRMVCQ WTQTRRRARN IPLEELQRNL QFHAFISYSE HDSAWVKSEL
VPYLEKEDIQ ICLHERNFVP GKSIVENIIN CIEKSYKSIF VLSPNFVQSE WCHYELYFAH
HNLFHEGSNN LILILLEPIP QNSIPNKYHK LKALMTQRTY LQWPKEKSKR GLFWANIRAA
FNMKLTLVTE NNDVKS
TLR7 (toll-like receptor 7 precursor) - NCBI Reference Sequence:
NP_057646.1
(SEQ ID NO:21)
MVFPMWTLKR QILILFNIIL ISKLLGARWF PKTLPCDVTL DVPKNHVIVD CTDKHLTEIP
GGIPTNTTNL TLTINHIPDI SPASFHRLDH LVEIDFRCNC VPIPLGSKNN MCIKRLQIKP
RSFSGLTYLK SLYLDGNQLL EIPQGLPPSL QLLSLEANNI FSIRKENLTE LANIEILYLG
QNCYYRNPCY VSYSIEKDAF LNLTKLKVLS LKDNNVTAVP TVLPSTLTEL YLYNNMIAKI
QEDDFNNLNQ LQILDLSGNC PRCYNAPFPC APCKNNSPLQ IPVNAFDALT ELKVLRLHSN
SLQHVPPRWF KNINKLQELD LSQNFLAKEI GDAKFLHFLP SLIQLDLSFN FELQVYRASM
NLSQAFSSLK SLKILRIRGY VFKELKSFNL SPLHNLQNLE VLDLGTNFIK IANLSMFKQF
KRLKVIDLSV NKISPSGDSS EVGFCSNART SVESYEPQVL EQLHYFRYDK YARSCRFKNK
EASFMSVNES CYKYGQTLDL SKNSIFFVKS SDFQHLSFLK CLNLSGNLIS QTLNGSEFQP
LAELRYLDFS NNRLDLLHST AFEELHKLEV LDISSNSHYF QSEGITHMLN FTKNLKVLQK
LMMNDNDISS STSRTMESES LRTLEFRGNH LDVLWREGDN RYLQLFKNLL KLEELDISKN
SLSFLPSGVF DGMPPNLKNL SLAKNGLKSF SWKKLQCLKN LETLDLSHNQ LTTVPERLSN
CSRSLKNLIL KNNQIRSLTK YFLQDAFQLR YLDLSSNKIQ MIQKTSFPEN VLNNLKMLLL
HHNRFLCTCD AVWFVWWVNH TEVTIPYLAT DVTCVGPGAH KGQSVISLDL YTCELDLTNL
ILFSLSISVS LFLMVMMTAS HLYFWDVWYI YHFCKAKIKG YQRLISPDCC YDAFIVYDTK
DPAVTEWVLA ELVAKLEDPR EKHFNLCLEE RDWLPGQPVL ENLSQSIQLS KKTVFVMTDK
YAKTENFKIA FYLSHQRLMD EKVDVIILIF LEKPFQKSKF LQLRKRLCGS SVLEWPTNPQ
AHPYFWQCLK NALATDNHVA YSQVFKETV
TLR8 (toll-like receptor 8 isoform 1) - UniProtKB/Swiss-Prot:
Q9NR97.1
(SEQ ID NO:22)
MENMFLQSSM LTCIFLLISG SCELCAEENF SRSYPCDEKK QNDSVIAECS NRRLQEVPQT
VGKYVTELDL SDNFITHITN ESFQGLQNLT KINLNHNPNV QHQNGNPGIQ SNGLNITDGA
FLNLKNLREL LLEDNQLPQI PSGLPESLTE LSLIQNNIYN ITKEGISRLI NLKNLYLAWN
CYFNKVCEKT NIEDGVFETL TNLELLSLSF NSLSHVPPKL PSSLRKLFLS NTQIKYISEE
DFKGLINLTL LDLSGNCPRC FNAPFPCVPC DGGASINIDR FAFQNLTQLR YLNLSSTSLR
KINAAWFKNM PHLKVLDLEF NYLVGEIASG AFLTMLPRLE ILDLSFNYIK GSYPQHINIS
RNFSKLLSLR ALHLRGYVFQ ELREDDFQPL MQLPNLSTIN LGINFIKQID FKLFQNFSNL
EIIYLSENRI SPLVKDTRQS YANSSSFQRH IRKRRSTDFE FDPHSNFYHF TRPLIKPQCA
AYGKALDLSL NSIFFIGPNQ FENLPDIACL NLSANSNAQV LSGTEFSAIP HVKYLDLTNN
RLDFDNASAL TELSDLEVLD LSYNSHYFRI AGVTHHLEFI QNFTNLKVLN LSHNNIYTLT
DKYNLESKSL VELVFSGNRL DILWNDDDNR YISIFKGLKN LTRLDLSLNR LKHIPNEAFL
NLPASLTELH INDNMLKFFN WTLLQQFPRL ELLDLRGNKL LFLTDSLSDF TSSLRTLLLS
HNRISHLPSG FLSEVSSLKH LDLSSNLLKT INKSALETKT TTKLSMLELH GNPFECTCDI
GDFRRWMDEH LNVKIPRLVD VICASPGDQR GKSIVSLELT TCVSDVTAVI LFFFTFFITT
MVMLAALAHH LFYWDVWFIY NVCLAKVKGY RSLSTSQTFY DAYISYDTKD ASVTDWVINE
LRYHLEESRD KNVLLCLEER DWDPGLAIID NLMQSINQSK KTVFVLTKKY AKSWNFKTAF
YLALQRLMDE NMDVIIFILL EPVLQHSQYL RLRQRICKSS ILQWPDNPKA EGLFWQTLRN
VVLTENDSRY NNMYVDSIKQ Y
TLR9 (toll-like receptor 9 precursor) - NCBI Reference Sequence:
NP_059138.1
(SEQ ID NO:23)
MGFCRSALHP LSLLVQAIML AMTLALGTLP AFLPCELQPH GLVNCNWLFL KSVPHFSMAA
PRGNVTSLSL SSNRIHHLHD SDFAHLPSLR HLNLKWNCPP VGLSPMHFPC HMTIEPSTFL
AVPTLEELNL SYNNIMTVPA LPKSLISLSL SHTNILMLDS ASLAGLHALR FLFMDGNCYY
KNPCRQALEV APGALLGLGN LTHLSLKYNN LTVVPRNLPS SLEYLLLSYN RIVKLAPEDL
ANLTALRVLD VGGNCRRCDH APNPCMECPR HFPQLHPDTF SHLSRLEGLV LKDSSLSWLN
ASWFRGLGNL RVLDLSENFL YKCITKTKAF QGLTQLRKLN LSFNYQKRVS FAHLSLAPSF
GSLVALKELD MHGIFFRSLD ETTLRPLARL PMLQTLRLQM NFINQAQLGI FRAFPGLRYV
DLSDNRISGA SELTATMGEA DGGEKVWLQP GDLAPAPVDT PSSEDFRPNC STLNFTLDLS
RNNLVTVQPE MFAQLSHLQC LRLSHNCISQ AVNGSQFLPL TGLQVLDLSH NKLDLYHEHS
FTELPRLEAL DLSYNSQPFG MQGVGHNFSF VAHLRTLRHL SLAHNNIHSQ VSQQLCSTSL
RALDFSGNAL GHMWAEGDLY LHFFQGLSGL IWLDLSQNRL HTLLPQTLRN LPKSLQVLRL
RDNYLAFFKW WSLHFLPKLE VLDLAGNQLK ALTNGSLPAG TRLRRLDVSC NSISFVAPGF
FSKAKELREL NLSANALKTV DHSWFGPLAS ALQILDVSAN PLHCACGAAF MDFLLEVQAA
VPGLPSRVKC GSPGQLQGLS IFAQDLRLCL DEALSWDCFA LSLLAVALGL GVPMLHHLCG
WDLWYCFHLC LAWLPWRGRQ SGRDEDALPY DAFVVFDKTQ SAVADWVYNE LRGQLEECRG
RWALRLCLEE RDWLPGKTLF ENLWASVYGS RKTLFVLAHT DRVSGLLRAS FLLAQQRLLE
DRKDVVVLVI LSPDGRRSRY VRLRQRLCRQ SVLLWPHQPS GQRSFWAQLG MALTRDNHHF
YNRNFCQGPT AE
TLR10 (toll-like receptor 10 isoform a) - NCBI Reference Sequence:
NP_001017388.1
(SEQ ID NO:24)
MRLIRNIYIF CSIVMTAEGD APELPEEREL MTNCSNMSLR KVPADLTPAT TTLDLSYNLL
FQLQSSDFHS VSKLRVLILC HNRIQQLDLK TFEFNKELRY LDLSNNRLKS VTWYLLAGLR
YLDLSFNDFD TMPICEEAGN MSHLEILGLS GAKIQKSDFQ KIAHLHLNTV FLGFRTLPHY
EEGSLPILNT TKLHIVLPMD TNFWVLLRDG IKTSKILEMT NIDGKSQFVS YEMQRNLSLE
NAKTSVLLLN KVDLLWDDLF LILQFVWHTS VEHFQIRNVT FGGKAYLDHN SFDYSNTVMR
TIKLEHVHFR VFYIQQDKIY LLLTKMDIEN LTISNAQMPH MLFPNYPTKF QYLNFANNIL
TDELFKRTIQ LPHLKTLILN GNKLETLSLV SCFANNTPLE HLDLSQNLLQ HKNDENCSWP
ETVVNMNLSY NKLSDSVFRC LPKSIQILDL NNNQIQTVPK ETIHLMALRE LNIAFNFLTD
LPGCSHFSRL SVLNIEMNFI LSPSLDFVQS CQEVKTLNAG RNPFRCTCEL KNFIQLETYS
EVMMVGWSDS YTCEYPLNLR GTRLKDVHLH ELSCNTALLI VTIVVIMLVL GLAVAFCCLH
FDLPWYLRML GQCTQTWHRV RKTTQEQLKR NVRFHAFISY SEHDSLWVKN ELIPNLEKED
GSILICLYES YFDPGKSISE NIVSFIEKSY KSIFVLSPNF VQNEWCHYEF YFAHHNLFHE
NSDHIILILL EPIPFYCIPT RYHKLKALLE KKAYLEWPKD RRKCGLFWAN LRAAINVNVL
ATREMYELQT FTELNEESRG STISLMRTDC L

The MTMs of the invention also include other TLR-based molecules that bind to one or more MAMPs, e.g. those MTMs comprising at least a portion (e.g. domain) of a TLR-based molecule in the case of an engineered MTM. As used herein, the term “TLR-based molecule” refers to a molecule comprising a microbe-binding domain (i.e. an N-terminal ligand-binding domain) derived from a TLR. The term “TLR” as used herein refers to any molecule including proteins, natural or genetically modified (e.g., recombinant), that interacts specifically with an MAMP and that has a Toll IL-1 receptor (TIR) domain in their signaling domain. The term “TLR” as used herein can also refer to TLR derived from any species, including, but not limited to, plants, animals (e.g. mammals, such as human), insects and microorganisms, having the desired binding specificity.

TLR-based engineered MTMs of the invention are MTMs that comprise at least a microbe-binding domain of a TLR, e.g. the N-terminal ligand-binding domain of a TLR. These MTMs may also include one or more of the other domains of a TLR, e.g. a transmembrane helix and/or a C-terminal cytoplasmic signaling domain, as well as one or more domains not typically found in a TLR, such as an oligomerization domain, a signal domain, an anchor domain, a collagen-like domain, a fibrinogen-like domain, an immunoglobulin domain, and/or an immunoglobulin-like domain. When a TLR-based engineered MTM has each of the domains of a wild-type TLR, the MTM will be a sequence-variant engineered MTM as defined above. When a TLR-based engineered MTM has fewer that all of the domains of a wild-type TLR, the MTM will be a domain-variant engineered MTM or a sequence- and domain-variant engineered MTM as defined above.

TLR-based engineered MTMs comprise a microbe-binding domain comprising at least one N-terminal ligand-binding domain of a TLR selected from the group consisting of TLR1, TLR2, TLR3, TLR4, TLR5, TLR6, TLR7, TLR8, TLR9, and TLR10.

In some aspects, the MTMs and engineered MTMs of the invention comprise a microbe-binding domain comprising the N-terminal ligand-binding domain of TLR1 of SEQ ID NO:15, or the N-terminal ligand-binding domain of TLR2 of SEQ ID NO:16, or the N-terminal ligand-binding domain of TLR3 of SEQ ID NO:17, or the N-terminal ligand-binding domain of TLR4 of SEQ ID NO:18, or the N-terminal ligand-binding domain of TLR5 of SEQ ID NO:19, or the N-terminal ligand-binding domain of TLR6 of SEQ ID NO:20, or the N-terminal ligand-binding domain of TLR7 of SEQ ID NO:21, or the N-terminal ligand-binding domain of TLR8 of SEQ ID NO:22, or the N-terminal ligand-binding domain of TLR9 of SEQ ID NO:23, or the N-terminal ligand-binding domain of TLR10 of SEQ ID NO:24.

In some aspects, the microbe-binding domain comprising a N-terminal ligand-binding domain of a TLR from a primate, mouse, rat, hamster, rabbit, or any other subject as described herein.

The exemplary sequences provided herein for the TLRs are not to be construed as limiting. For example, while the exemplary sequences provided herein are derived from a human, amino acid sequences of TLRs from other species such as mice, rats, porcine, bovine, feline, and canine are known in the art and within the scope described herein.

An exemplary MTM may include a microbe surface-binding domain. The microbe surface-binding domain can include a mannose-binding lectin (MBL). The microbe surface-binding domain may comprise a human mannose-binding lectin (MBL). The microbe surface-binding domain can include a carbohydrate recognition domain (CRD) of MBL. The CRD may be linked to an immunoglobulin or fragment thereof. In certain aspects, the CRD may be linked to an Fc component of human IgG1 (FcMBL). Any known engineered microbe or microbe component-binding molecule can be used to bind target microbes or components including MTMs comprising a binding domain such as an Fc Lectin, Fc-Collagen-CRD, Collagen-Fc-CRD, Collagen-CRD-Fc, FcCD209 (DC-SIGN), Fc209L, FcCD14 (LPS-binding protein), FcPGRP (Peptidoglycan recognition proteins), FcCRP (C-Reactive Protein), a lectin targeting Protein-A expressing microbes, or a lectin targeting Protein-G expressing microbes. Descriptions and sequences of various engineered proteins and component microbe surface-binding domains thereof contemplated for use with systems and methods of the invention are provided in U.S. Pat. No. 9,150,631; U.S. Ser. No. 15/105,298; U.S. Ser. No. 16/059,799; U.S. Pat. Nos. 9,593,160; 10,435,457 and U.S. Pat. Pub. 2015/0173883, the contents of each of which are incorporated herein by reference.

### Methods

A method of preparing a sample for detecting a microbe or microbe components present in the sample can comprise adding a substance to a sample suspected of comprising a microbe or microbe components, digesting the sample under conditions promoting digestion of a microbe or microbe components, and optionally contacting the digested microbe or microbe components with a matrix or matrix solution on a target substrate.

In some aspects, the method can further comprise, prior to adding the substance, culturing the sample to grow the microbe or microbe components. In some aspects, the method can further comprise, prior to adding the substance, contacting the sample with an MTM bound substrate and isolating from a sample a microbe or microbe components bound to an MTM on a substrate.

Microbe isolation and analysis may be performed on whole, intact microbes or any portion or subpart thereof (e.g., cell wall components, outer membranes, nucleic acid (e.g., DNA, including 16S ribosomal DNA, and RNA), plasma membranes, ribosomes, microbial capsule, pili, or flagella. Microbe isolation and analysis as described herein may also involve identification of microbe-associated molecular patterns (MAMPs), pathogen-associated molecular patterns (PAMPs), and/or microbe-associated proteins.

The isolating step may be in accordance with a characteristic of the target microbe or microbe component or of the substrate or other bound capture molecule. Exemplary characteristics used for isolation may include size, mass, density, charge. In some aspects, the sample can be contacted with MTMs linked to a substrate. The engineered molecule can be a protein with engineered specificity for a particular microbe, microbe component, or class of either. The substrate to which the engineered molecules are linked or coupled can be an interior surface of a flow-through column, a bead, a magnetic particle, or any other known substrate used in target capture and separation. In certain aspects, the substrate may be a magnetic substrate or ELISA plate.

The step of isolating may include applying a magnet to the sample. For example, the engineered molecules described above may be linked to a magnetic particle such that application of a magnetic field to the sample can isolate the magnetic particles as well as the linked engineered molecule and any microbe or components bound thereto. Methods of the invention may use a superparamagnetic substrate. The magnetic substrate may comprise at least one of a magnetic bead, a superparamagnetic bead, or a magnetic microbead. In certain aspects, the MTM may be linked to an ELISA plate. Examples of magnetic capture techniques as well as ELISA-related substrates compatible with methods of the invention are described, for example, in U.S. Pat. Pub. 2015/0173883, already incorporated by reference in its entirety herein.

In certain aspects, isolating may include concentrating the microbe or microbe components of the sample. For example, after binding the target microbes or components thereof to a substrate using the engineered targeting molecules, the remaining sample, along with any unbound molecules can continue to flow out of the capture device or other substrate. The captured microbes or components thereof can then be washed in one or more steps to further remove unbound material. In various aspects, wash fluids may include calcium. The removal of unbound material allows for focused analysis of the target microbes and reduces the overall sample volume before any analysis steps.

To aid in binding of the microbe targeting molecule to the target microbe or microbe component and/or to prevent off-target binding and remove unbound material, the sample may be agitated and optionally heated. For example, the sample may be held at about 20° C. or more for about 1 minute or more, for example, up to about 20 minutes or up to about 30 minutes, to allow for microbe or microbe component binding.

Isolation can include elution of the microbe to release them from the bound substrate for further analysis. For example, after washing or otherwise removing unbound sample material, the remaining, captured target microbes can be eluted through a variety of known methods to allow for subsequent analysis steps without interference of the binding substrate or engineered targeting molecules. Elution may be accomplished through any known means and will generally depend on the desired analysis method and the composition of the substrate and engineered targeting molecule. Exemplary elution methods include temperature-based (e.g., heating to 70° C. or more), physical (e.g., agitation), photosensitive cleavage, or chemical methods. In certain aspects, elution through heating may be performed in calcium-free water. Exemplary chemical elution methods may involve a change in pH and/or application of a chelation agent. Chelation agents may include one or more of ethylenediaminetetraacetic acid (EDTA), calcium disodium edetate (CaNa₂EDTA), ethylene glycol-bis(3-aminoethyl ether)-N,N,N′,N′-tetraacetic acid (EGTA), 1,2-bis(o-aminophenoxy)ethane-N,N,N′,N′-tetraacetic acid (BAPTA), deferoxamine mesylate salt (DFOM).

After isolation of microbes or microbe components from the sample, the captured material can be digested for analysis. Digestion can refer to the release of constituent microbial components for subsequent analysis. Digestion can occur through exposure to a substance selected based on the desired analysis method and the target microbe to analyzed. In some aspects, that lysing or killing microbes in a sample by mechanical treatment (e.g., beadmilling, sonication, or other functionally equivalent method to disrupt cell wall), and/or chemical treatment (e.g., antibiotics, antivirals, antifungals or other antimicrobial agents) can allow detection of encapsulated microbes such as Klebsiella species that would not be otherwise detected. Thus, a pre-treatment of a sample to lyse or kill microbes can be performed prior to binding of the microbe-targeting molecules to exposed MAMPs. Therefore, this will not only increase the sensitivity of a microbe-targeting molecule-based detection method but can also surprisingly and significantly increase the spectrum of microbes that can be detected by an MTM-based detection method.

In some aspects, the patient has been treated with at least one antimicrobial agent. In some aspects, the sample contains at least one antibiotic or at least one antimicrobial agent, non-limiting examples of which are described further herein. In some aspects, the sample contains at least two antibiotics or at least two antimicrobial agents.

In some aspects, the patient has been treated with antibiotics, non-limiting examples of which are described further herein. In some aspects, the sample contains antibiotics, for example at least 1, at least 2, at least 3, at least 4, or at least 5 antibiotics.

In some aspects, the patient has been treated with antifungals, non-limiting examples of which are described further herein. In some aspects, the sample contains antifungals, for example at least 1, at least 2, at least 3, at least 4, or at least 5 antifungals.

In some aspects, the patient has been treated with antivirals, non-limiting examples of which are described further herein. In some aspects, the sample contains antivirals, for example at least 1, at least 2, at least 3, at least 4, or at least 5 antivirals.

Antibiotics can be from classes including Cephalosporin, Glycopeptide, Cyclic lipopeptide, Aminoglycoside, Macrolide, Oxazolidinone, Fluoroquinolones, Lincosamides, or Carbapenem. Antifungals can be from classes including Polyenes, Azoles, Nucleoside Analog, Echinocandin, or Allylamine. Antivirals can be from classes including CCR5 anatonists, Fusion inhibitors, Nucleoside/Nucleotide reverse transcriptase inhibitors (NRTIs), Non-nucleoside reverse transcriptase inhibitors (NNRTIs), Nucleotide reverse transcriptase inhibitors (NtRTIs), Integrase inhibitors, Protease inhibitors, DNA polymerase inhibitors, Guanosine analogs, Interferon-alpha, M2 ion channel blockers, Nucleoside inhibitors, NS5A polymerase inhibitors, NS3/4A protease inhibitors, Neuraminidase inhibitors, Nucleoside analogs, and Direct acting antivirals (DAAs).

In some aspects, the antimicrobial agent can be selected from aminoglycosides, ansamycins, beta-lactams, bis-biguanides, carbacephems, carbapenems, cationic polypeptides, cephalosporins, fluoroquinolones, glycopeptides, iron-sequestering glycoproteins, linosamides, lipopeptides, macrolides, monobactams, nitrofurans, oxazolidinones, penicillins, polypeptides, quaternary ammonium compounds, quinolones, silver compounds, sulfonamides, tetracyclines, and any combinations thereof. In some aspects, the antimicrobial agent can comprise an antibiotic.

Some exemplary specific antimicrobial agents include broad penicillins, amoxicillin (e.g., Ampicillin, Bacampicillin, Carbenicillin Indanyl, Mezlocillin, Piperacillin, Ticarcillin), Penicillins and Beta Lactamase Inhibitors (e.g., Amoxicillin-Clavulanic Acid, Ampicillin-Sulbactam, Benzylpenicillin, Cloxacillin, Dicloxacillin, Methicillin, Oxacillin, Penicillin G, Penicillin V, Piperacillin Tazobactam, Ticarcillin Clavulanic Acid, Nafcillin), Cephalosporins (e.g., Cephalosporin I Generation, Cefadroxil, Cefazolin, Cephalexin, Cephalothin, Cephapirin, Cephradine), Cephalosporin II Generation (e.g., Cefaclor, Cefamandole, Cefonicid, Cefotetan, Cefoxitin, Cefprozil, Cefmetazole, Cefuroxime, Loracarbef), Cephalosporin III Generation (e.g., Cefdinir, Ceftibuten, Cefoperazone, Cefixime, Cefotaxime, Cefpodoxime proxetil, Ceftazidime, Ceftizoxime, Ceftriaxone), Cephalosporin IV Generation (e.g., Cefepime), Macrolides and Lincosamides (e.g., Azithromycin, Clarithromycin, Clindamycin, Dirithromycin, Erythromycin, Lincomycin, Troleandomycin), Quinolones and Fluoroquinolones (e.g., Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, Perfloxacin), Carbapenems (e.g., Imipenem-Cilastatin, Meropenem), Monobactams (e.g., Aztreonam), Aminoglycosides (e.g., Amikacin, Gentamicin, Kanamycin, Neomycin, Netilmicin, Streptomycin, Tobramycin, Paromomycin), Glycopeptides (e.g., Teicoplanin, Vancomycin), Tetracyclines (e.g., Demeclocycline, Doxycycline, Methacycline, Minocycline, Oxytetracycline, Tetracycline, Chlortetracycline), Sulfonamides (e.g., Mafenide, Silver Sulfadiazine, Sulfacetamide, Sulfadiazine, Sulfamethoxazole, Sulfasalazine, Sulfisoxazole, Trimethoprim-Sulfamethoxazole, Sulfamethizole), Rifampin (e.g., Rifabutin, Rifampin, Rifapentine), Oxazolidinones (e.g., Linezolid, Streptogramins, Quinupristin Dalfopristin), Bacitracin, Chloramphenicol, Fosfomycin, Isoniazid, Methenamine, Metronidazole, Mupirocin, Nitrofurantoin, Nitrofurazone, Novobiocin, Polymyxin, Spectinomycin, Trimethoprim, Colistin, Cycloserine, Capreomycin, Ethionamide, Pyrazinamide, Para-aminosalicylic acid, Erythromycin ethylsuccinate, and the like.

Some exemplary antifungals include polyene antifungals, Amphotericin B, Candicidin, Filipin, Hamycin, Natamycin, Nystatin, Rimocidin, imidazole antifungals, triazole antifungals, thiazole antifungals, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Triazoles, Albaconazole, Efinaconazole, Epoxiconazole, Fluconazole, Isavuconazole, Itraconazole, Posaconazole, Propiconazole, Ravuconazole, Terconazole, Voriconazole, Abafungin, Allylamines, amorolfin, butenafine, naftifine, terbinafine, Echinocandins, Anidulafungin, Caspofungin, Micafungin, Aurones, Benzoic acid, Ciclopirox, Flucytosine, 5-fluorocytosin, Griseofulvin, Haloprogin, Tolnaftate, Undecylenic acid, Triacetin, Crystal violet, Castellani's paint, Orotomide, Miltefosine, Potassium iodide, Coal tar, Copper(II) sulfate, Selenium disulfide, Sodium thiosulfate, Piroctone olamine, Iodoquinol, clioquinol, Acrisorcin, Zinc pyrithione, and Sulfur. Additional antifungals known in the art can also be used.

Some exemplary antivirals agents include Abacavir, Acyclovir, Adefovir, Amantadine, Ampligen, Amprenavir, antiretroviral, Arbidol, Atazanavir, Atripla, Boceprevir, Cidofovir, Combivir, Daclatasvir, Darunavir, Delavirdine, Dasabuvir, Didanosine, Docosanol, Dolutegravir, Doravirine, Ecoliever, Edoxudine, Efavirenz, Elbasvir, Emtricitabine, Enfuvirtide, Entecavir, Etravirine, Famciclovir, Fomivirsen, Fosamprenavir, Foscarnet, Fosfonet, Fusion inhibitor, Ganciclovir, Gemcitabine, Glecaprevir, Grazoprevir, Ibacitabine, Idoxuridine, Imiquimod, Imunovir, Indinavir, Inosine, Integrase inhibitor, Interferon, Interferon type I, Interferon type II, Interferon type III, Lamivudine, Ledipasvir, Lopinavir, Lopiravir, Loviride, Maraviroc, Methisazone, Moroxydine, Nelfinavir, Nevirapine, Nexavir, Nitazoxanide, Norvir, Nucleoside analogues, Ombitasvir, Oseltamivir (Tamiflu), Paritaprevir, Peglyated Interferon-alpha, Peginterferon alfa-2a, Penciclovir, Peramivir, Pibrentasvir, Pleconaril, Podophyllotoxin, Protease inhibitor, Pyramidine, Raltegravir, Reverse transcriptase inhibitor, Ribavirin, Rilpivirine, Rimantadine, Ritonavir, Saquinavir, Simeprevir, Sofosbuvir, Stavudine, Synergistic enhancer (antiretroviral), Telaprevir, Telbivudine, Tenofovir, Tenofovir disoproxil, Tipranavir, Trifluridine, Trizivir, Tromantadine, Truvada, Valaciclovir (Valtrex), Valganciclovir, Velpatasvir, Vicriviroc, Vidarabine, Viramidine, Voxilaprevir, Zalcitabine, Zanamivir (Relenza), Zidovudine. Additional antivirals known in the art can also be used.

Without limitations, incubation of microbes present in the sample with one or more antimicrobial agents can be at any desired temperature and for any desired duration. In some aspects, the incubation can be performed at room temperature or at an elevated temperature. In some aspects, incubation can be performed at a temperature of about 30° C. to about 45° C. In one aspect, incubation can be performed at a temperature of about 37° C.

As indicated above, incubation of microbes present in a sample can be performed for any desired time period, which can vary with a number of factors, including but not limited to, temperature of incubation, concentration of microbes in the sample, and/or potency and/or concentrations of antimicrobial agents used. In some aspects, incubation can be for about at least one minute (e.g. one, five, ten, fifteen, twenty, twenty-five, thirty, thirty-five, forty, forty-five, fifty-five, sixty, ninety minutes or more). In some aspects, incubation can be for at least about one hour, at least about two hours, at least about three hours, at least about four hours, at least about five hours, at least about six hours, at least about seven hours, at least about eight hours, at least about nine hours, at least about ten hours or more. In some aspects, incubation can be for a period of about fifteen minutes to about ninety minutes. In one aspect, incubation can be for a period of about thirty minutes to about sixty minutes. In another aspect, incubation can be for a period of about thirty minutes to about twenty-four hours. In one aspect, incubation can be for a period of at least about four hours.

In some aspects, the pre-treatment can comprise incubating the sample with at least one or more degradative enzymes. For example, in some aspects, a degradative enzyme can be selected to cleave at least some of the cell wall carbohydrates, thus restoring detection of carbohydrates that are otherwise not recognized by MTMs. In some aspects, a degradative enzyme can be selected to cause call wall degradation and thus release or expose MAMPs that are otherwise unable bind to the MTMs. Other examples of degradative enzymes include, but are not limited to, proteases, lipases such as phospholipases, neuraminidase, and/or sialidase, or any other enzyme modifying the presentation of any MAMP to any MTM leveraged for detection of the MAMP. For instance, an exemplary MTM can comprise MBL or recombinant human MBL or engineered FcMBL, which binds mannose containing carbohydrates such as the core of LPS, the Wall Teichoic Acid from Staphylococcus aureus, PIM6 or Mannose-capped LipoArabinoMannan from M. tuberculosis whereas CRP binds phosphocholine found in Streptococcus pneumonia (Brundish and Baddiley, 1968), Haemophilus influenzae (Weiser et al., 1997), Pseudomonas aeruginosa, Neisseria meningitides, Neisseria gonorrhoeae (Serino and Virji, 2000), Morganella morganii (Potter, 1971), and Aspergillus fumigatus (Volanakis, “Human C-reactive protein: expression, structure, and function, “Molecular Immunology,” 2001, 38(2-3): 189-197). Other MTMs can be equally leveraged to recognize MAMPs such as nucleotide-binding oligomerization domains (NODs) or peptidoglycan recognition proteins (PGRP).

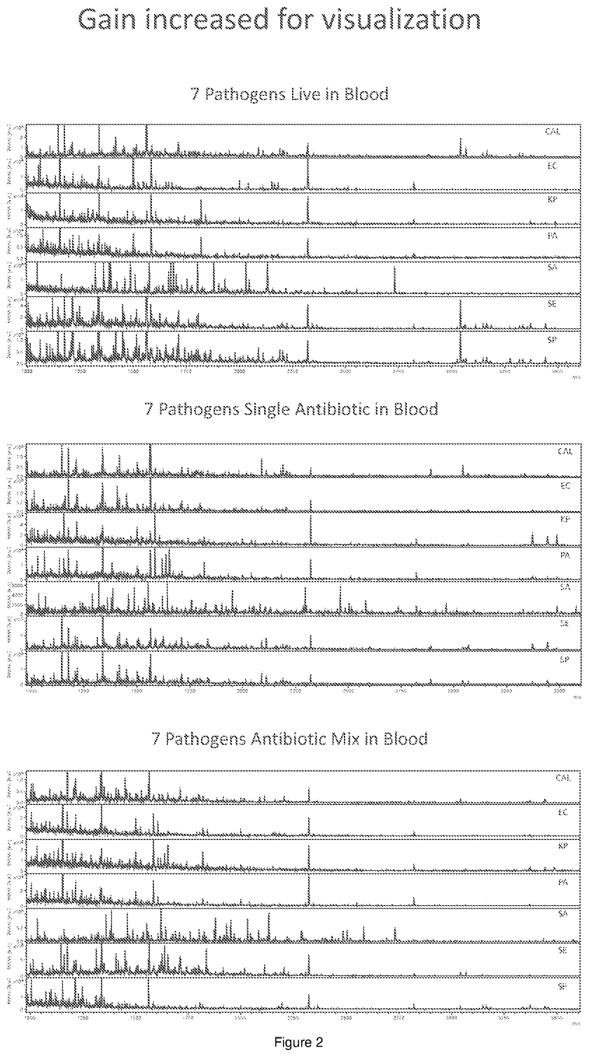

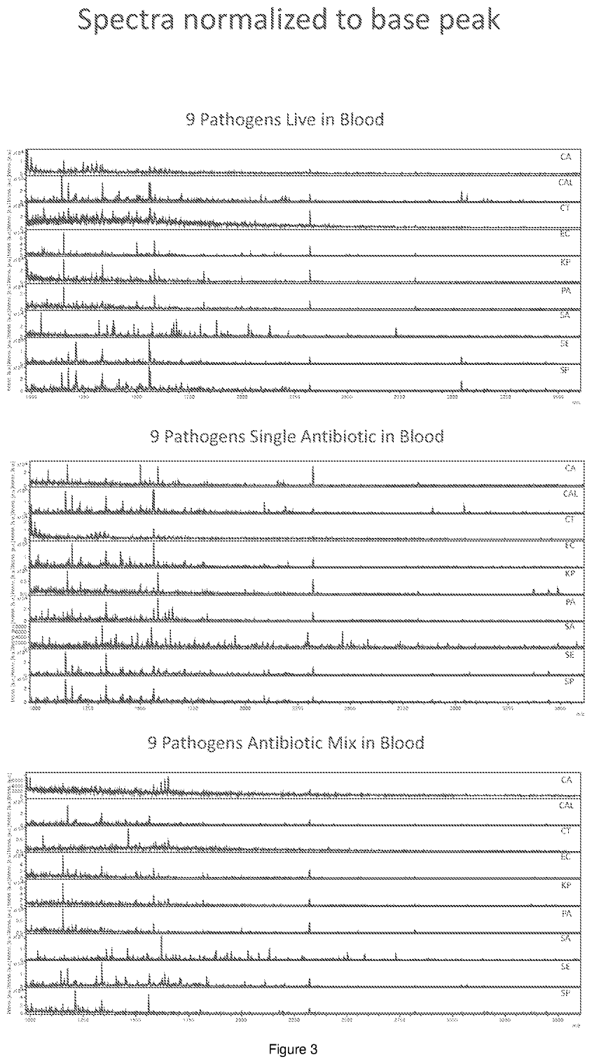

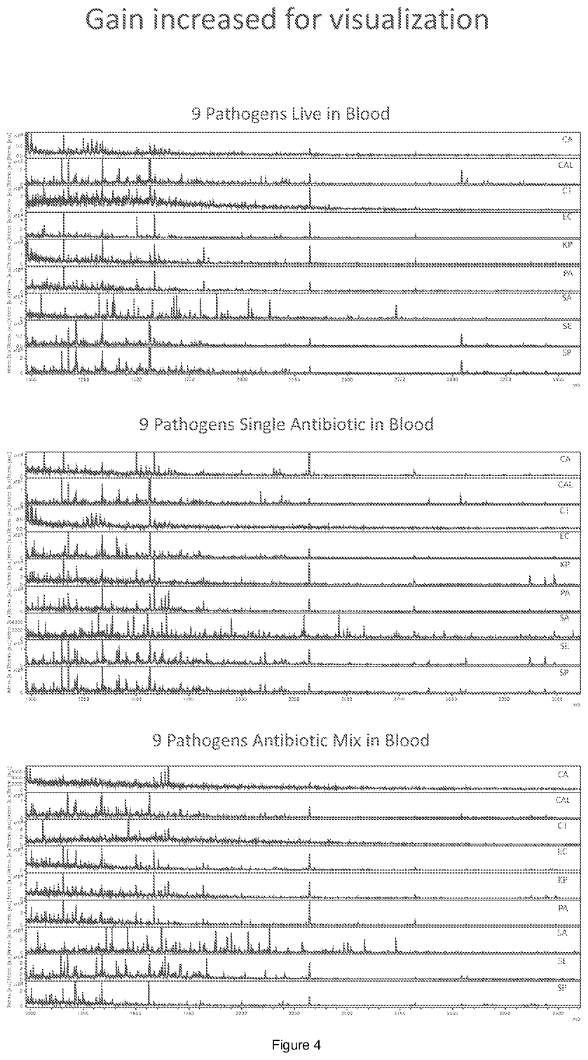

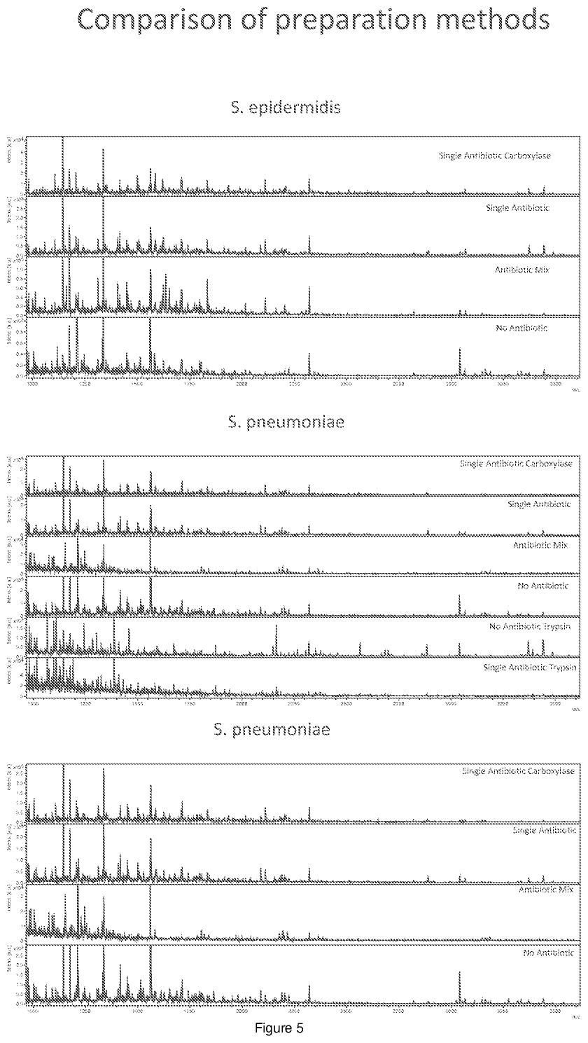

In some aspects, an antimicrobial mixture can be added during the digestion step where the antimicrobial mixture can include one or more antibiotics and/or one or more antifungals and/or one or more antivirals. Digesting the sample with a single antimicrobial, while enhancing the spectra, may cause variation in the spectra for a single pathogen between antimicrobial classes administered. Therefore, digesting the sample with an antimicrobial mixture, a normalized spectrum for each pathogen may be obtained, as shown in FIGS. 1-5.

In some aspects, the antimicrobial mixture may include one or more classes of antimicrobials including but not limited to: Cephalosporin, Glycopeptide, Cyclic lipopeptide, Aminoglycoside, Macrolide, Oxazolidinone, Fluoroquinolone, Lincosamide, Carbapenem; Ecuhocandcin, or Polyene.

In some aspects, the antimicrobial mixture may include one or more of cefepime, vancomycin, daptomycin, amikacin, erythromycin, linezolid, ciproflaxin, lincomycin, meropenem, caspofungin or amphotericin. In a nonlimiting example, the antimicrobial mixture may include cefepime, vancomycin, daptomycin, amikacin, erythromycin, linezolid, ciproflaxin, lincomycin, meropenem, caspofungin and amphotericin. In another nonlimiting example, the antimicrobial mixture may include cefepime, vancomycin, daptomycin, amikacin, erythromycin, linezolid, ciproflaxin, lincomycin, and meropenem. In another nonlimiting example, the antimicrobial mixture may include caspofungin and amphotericin.

The antimicrobial mixture can include an antibiotic mixture at a concentration from about 0.1 ug/mL to about 100 mg/mL. The antimicrobial mixture may include an antifungal mixture at a concentration from about 0.01 ug/mL to about 100 mg/mL. The antimicrobial mixture may include an antiviral mixture at a concentration from about 0.01 ug/mL to about 100 mg/mL.

The amount of one or more antimicrobial agent added to a sample can be any desired amount and vary with a number of factors, including but not limited to, types of microbes in the sample, and/or potency of antimicrobial agents used. For example, one or more antimicrobial agents added to sample can have a concentration ranging from nanomolars to millimolars. In some aspects, one or more antimicrobial agents added to a sample can have a concentration ranging from 0.01 nM to about 100 mM, from about 0.01 nM to about 10 mM, or from about 0.1 nM to about 1 mM. In some aspects, one or more antimicrobial agents added to a sample can have a concentration ranging from nanograms per milliliters to micrograms per milliliters. In some aspects, one or more antimicrobial agents added to a sample can have a concentration ranging from about 1 ng/mL to about 1000 μg/mL, from about 10 ng/mL to about 750 μg/mL, or from about 100 ng/mL to about 500 μg/mL. In some aspects, one or more antimicrobial agents added to a sample can have a concentration ranging from about 10 μg/mL to about 500 μg/mL or from about 100 μg/mL to about 500 μg/mL.

Alternative to or in addition to the antimicrobial mixture, the substance used in digestion may include one or more enzymes, proteases, or carbohydrate-cleaving enzymes. In certain aspects, the substance used in the digestion can be trypsin. Digestion with a protease after isolation can standardize the isolated microbe or microbe components and can increase the probability of correctly identifying the microbe. In some aspects, the protease is selected from the group consisting of trypsin, chymotrypsin, pepsin, papain, elastase, or any combination thereof. The protease can also be any protease or protease mixture known in the art. Non-limiting examples of proteases include serine proteases, cysteine proteases, threonine proteases, aspartic proteases, glutamic proteases, metalloproteases, asparagine peptide lyases. In some aspects, the isolated microbe or microbe components are digested with at least one protease, at least 2 proteases, at least 3 proteases, at least 4 proteases, or at least 5 proteases, concurrently and/or sequentially. In some aspects, the protease is substantially free of protease inhibitors (e.g., 4-(2-Aminoethyl)benzenesulfonyl fluoride hydrochloride (AEBSF), Aprotinin, Bestatin, E64, Leupeptin, Pepstatin A).

In some aspects, the protease is trypsin. It is noted that trypsin is not commonly used in MALDI detection of microbes. In some aspects, the trypsin can be α-trypsin, β-trypsin, trypsin 1, trypsin 2, or mesotrypsin. In some aspects, the trypsin is at least 10% trypsin. As a non-limiting example, the trypsin is at least 1%, at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80%, or at least 90% trypsin. In some aspects, the trypsin is substantially free of trypsin inhibitors (e.g., Ca²⁺, Mg²⁺, heat, serpin, etc.). In some aspects, the trypsin comprises a divalent cation chelator (e.g., EDTA).

In some aspects, the isolated microbe or microbe component is digested for at most 30 seconds, at most 1 minute, at most 2 minutes, at most 3 minutes, at most 4 minutes, at most 5 minutes, at most 6 minutes, at most 7 minutes, at most 8 minutes, at most 9 minutes, at most 10 minutes, at most 20 minutes, at most 30 minutes, at most 40 minutes, at most 50 minutes, at most 60 minutes, at most 70 minutes, at most 80 minutes, at most 90 minutes, at most 2 hours, at most 3 hours, at most 4 hours, at most 5 hours, at most 6 hours, at most 7 hours, at most 8 hours, at most 9 hours, at most 10 hours, at most 11 hours, or at most 12 hours. In some aspects, the isolated microbe or microbe component is digested overnight.

In some aspects, the isolated microbe or microbe component is digested at human body temperature (e.g., 36-38° C.). In some aspects, the isolated microbe or microbe component is digested at a temperature that is greater than 36-38° C. In some aspects, the digestion of the isolated microbe or microbe component further comprises heating the digestion. For example, heating the protease can permit faster digestion and can increase the probability of correctly identifying the microbe.

In some aspects, heating the digestion comprises microwave treatment. In some aspects, the microwave treatment of the digestion is at a power of least 500 watts (W), at least 600 W, at least 700 W, at least 800 W, at least 900 W, at least 1000 W, at least 1100 W, at least 1200 W, at least 1300 W, at least 1400 W, or at least 1500 W. In some aspects, the microwave treatment of the digestion occurs for 1 minute. As a non-limiting example, the microwave treatment of the digestion can occur for at most 10 seconds, at most 20 seconds, at most 30 seconds, at most 40 seconds, at most 50 seconds, at most 1 minute, at most 2 minutes, at most 3 minutes, at most 4 minutes, at most 5 minutes, at most 6 minutes, at most 7 minutes, at most 8 minutes, at most 9 minutes, or at most 10 minutes.

In some aspects, the method described herein further comprises contacting the digested microbe or microbe components with a composition that is more acidic than the digested microbe or microbe components (e.g., said step of contacting can decrease the pH of the solution). As used herein, “more acidic” refers to a composition or solution with a lower pH compared to another composition or solution. Contacting the digested microbe or microbe components with such a composition can quickly and effectively quench the protease digestion reaction, increase component stability, and improve mass spectrometry (e.g., MALDI) sensitivity.

In some aspects, the composition that is more acidic than the digested microbe or microbe components is present at a volume equal to or greater than the volume of the digested microbe or microbe components. As a non-limiting example, the volume of the composition that is more acidic than the digested microbe or microbe components can be present at a 1:1, 5:4, 4:3, 3:2, 2:1 ratio to the volume of the digested microbe or microbe components.

In some aspects, the composition that is more acidic than the digested microbe or microbe components is present at a concentration of at least 0.5%. As a non-limiting example, the concentration of the composition that is more acidic than the digested microbe or microbe components can be at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, at least 5.0%, at least 6.0%, at least 7.0%, at least 8.0%, at least 9.0%, or at least 10.0%.

In some aspects, the composition that is more acidic than the digested microbe or microbe components is selected from the group consisting of trifluoroacetic acid (TFA; CF₃COOH), acetic acid (CH₃COOH), and formic acid (CH₃COOH). As a non-limiting example, the composition that is more acidic than the digested microbe or microbe components can be hydrofluoric acid (HF), phosphoric acid (H₃PO₄), nitrous acid (HNO₂), lactic acid, citric acid, oxalic acid, uric acid, malic acid, or any carboxylic acid (—COOH). As a non-limiting example, the composition that is more acidic than the digested microbe or microbe components can be hydrochloric acid (HCl), nitric acid (HNO₃), —sulfuric acid (H₂SO₄), hydrobromic acid (HBr), hydroiodic acid (HI), perchloric acid (HClO₄), or chloric acid (HClO₃). As a non-limiting example, the composition that is more acidic than the digested microbe or microbe components can be any composition with a pH below 7.

In some aspects, the isolated microbe or microbe components are digested with a protease but not heated and not contacted with a composition that is more acidic than the digested microbe or microbe components. In some aspects, the isolated microbe or microbe components are digested with a protease and heated but not contacted with a composition that is more acidic than the digested microbe or microbe components. In some aspects, the isolated microbe or microbe components are digested with a protease and contacted with a composition that is more acidic than the digested microbe or microbe components but not heated. In some aspects, the isolated microbe or microbe components are digested with a protease, heated, and contacted with a composition that is more acidic than the digested microbe or microbe components. In some aspects, the isolated microbe or microbe components are not digested with a protease, not heated, and not contacted with a composition that is more acidic than the digested microbe or microbe components.

In some aspects, the sample has not been cultured. In other words, the microbes in the sample have not been allowed to replicate or amplify in a culture medium. Accordingly, in some aspects, the methods described herein do not comprise a culturing step, e.g., a step involving culturing and/or maintaining the microbe(s) ex vivo or in vitro. In some aspects, the time from the step of collecting the sample to the end of detection takes equal to or less than 90 minutes. As a non-limiting example, the time from the step of collecting the sample to the end of detection takes at most 60 minutes, at most 70 minutes, at most 80 minutes, at most 90 minutes, at most 100 minutes, at most 110 minutes, at most 120 minutes, at most 2.5 hours, at most 3.0 hours, at most 3.5 hours, at most 4.0 hours, at most 4.5 hours, at most 5.0 hours, at most 5.5 hours, at most 6.0 hours, at most 12.0 hours, at most 18 hours, or at most 24 hours.

In various aspects, microbes may be contacted with a matrix or matrix solution. The substrate can be evenly sprayed with matrix solution prior to analyzing the microbe or microbe components to generate a homogenous layer of crystallized matrix on top of the substrate. The application of a crystallized matrix can assist in certain analysis techniques including MALDI mass spectrometry. The desired matrix consists of crystallized molecules such as 3,5-dimethoxy-4-hydroxycinnamic acid (sinapinic acid), α-cyano-4-hydroxycinnamic acid (α-CHCA, alpha-cyano or alpha-matrix) and 2,5-dihydroxybenzoic acid (DHB). A solution of one of these molecules is made, often in a mixture of highly purified water and an organic solvent such as acetonitrile (ACN) or ethanol. Trifluoroacetic acid (TFA), as discussed above, can be used as a counter ion source. An exemplary matrix-solution is 20 mg/mL sinapinic acid in CAN at a ratio of 50:50:0.1 with water and TFA.

The application of a laser energy absorbing matrix in the sample preparation allows for the application of ionization-based analysis techniques (e.g., mass spectrometry) with minimal fragmentation. By applying a matrix directly to captured target microbes separated from sample, accurate microbe characterization can be carried out with minimal steps and delay. Accordingly, actionable results can be quickly obtained leading to quicker treatments and better patient outcomes. The use of a crystallized matrix is particularly useful in the analysis of biomolecules such as microbes and components thereof, which tend to be fragile and fragment when ionized by conventional ionization methods.

Methods of the invention may include analyzing the sample. Analysis may use volatile organic compound methods; Raman spectroscopy; FFT (Fast-Fourier Transform); Fourier-Transform Infrared Spectroscopy (FTIR); infrared spectrometry; Nuclear Magnetic Resonance (NMR) spectrometry; chromatographic methods; or mass spectrometric methods. In some aspects, the mass spectrometric method can comprises at least one of electron ionization, chemical ionization, electrospray ionization, atmospheric pressure chemical ionization, and matrix-assisted laser desorption/ionization time-of-flight mass (MALDI-TOF MS). Methods of mass spectrometry are known in the art and described in, for example, U.S. Pat. Nos. 8,895,918; 9,546,979; 9,761,426; Hoffman and Stroobant, Mass Spectrometry: Principles and Applications (2nd ed.). John Wiley and Sons (2001), ISBN 0-471-48566-7; Dass, Principles and practice of biological mass spectrometry, New York: John Wiley (2001) ISBN 0-471-33053-1; and Lee, ed., Mass Spectrometry Handbook, John Wiley and Sons, (2012) ISBN: 978-0-470-53673-5, the contents of each of which are incorporated herein by reference. Exemplary mass spectrometers are manufactured by Bruker. The mass spectrometric method can be automated.

Mass spectrometry or other analysis of captured microbes or microbial components can be used to identify the species and/or strain of microbe. Such identification is particularly useful where the microbe is a pathogen. Human pathogenic and other microbes or components thereof can be identified using mass spectrometry results as described in Singhai, et al., 2015, MALDI-TOF mass spectrometry: an emerging technology for microbial identification and diagnosis, Front Microbiol., 6:791, incorporated herein by reference.

In addition to identification of microbes or microbe components, mass spectrometry or other analysis of captured microbes or microbial components can be used to determine the antibiotic susceptibility of the captured microbe allowing infected patients to receive the most effective treatment with minimal delay, thereby reducing the risk of complications such as septic shock. For example, a method of determining an antimicrobial susceptibility of a microbe can include collecting at least one sample from a source comprising at least one microbe or microbe component, preparing the at least one sample, and performing an antimicrobial susceptibility test.

Preparing of the at least one sample, as described herein, can include contacting the sample with an MTM, for example, FcMBL, linked to a substrate, isolating the microbe or microbe components bound to the MTM, digesting the isolated microbe or microbe components with a substance, and contacting the microbe or microbe components with a matrix or matrix solution on a target substrate. In some aspects, the target substrate is evenly sprayed with matrix solution prior to analyzing microbe or microbe components to generate a homogenous layer of crystallized matrix on top of the substrate.

An antimicrobial susceptibility test can include obtaining a first signal from the sample comprising at least one microbe or microbe component, obtaining a second signal from the sample comprising the at least one microbe or microbe component and at least one antimicrobial; and comparing the first and second signal, where if the difference between the first and second signals is greater than a determined threshold, the at least one microbe is susceptible to the at least one antimicrobial, and wherein the first and second signals are obtained using a mass spectrometry method. The at least one antimicrobial is provided to the sample after the first signal is obtained.

The source can be a human, animal, plant, environment, organic material or inorganic material. The sample can be a bodily fluid of a human. The sample can include a buffer solution.

The method can include collecting a second sample from a human after the human is treated with at least one antimicrobial, where the second signal is obtained from the second sample, and where if the difference between the first and second signals is greater than a determined threshold, the at least one microbe is susceptible to the at least one antimicrobial. The second sample can be collected 24 hours or less after the first sample.

In some aspects, the second signal can be compared to a signal library, where the library comprises a signal profile for each of a plurality of microbes, and if the difference between the second signal and any of the plurality of microbe signal profiles is less than a determined threshold, the at least one microbe is suspectable to at least one antimicrobial identified in at least one of the plurality of microbe signal profiles. The first and second signals can be entered into a signal library.

The method can include inoculating the sample for 24 hours or less after the first signal is obtained and obtaining a third signal from the inoculated sample, where if the difference between the first and third signals is greater than a determined threshold, the sample comprises at least one live microbe. Additionally, if the difference between the first and second signals is greater than a determined threshold, the at least one microbe is susceptible to the at least one antimicrobial.

The method can include inoculating the sample for greater than 24 hours after the first signal is obtained and obtaining a third signal from the inoculated sample, where if the difference between the first and third signals is greater than a determined threshold, the sample comprises at least one live microbe. Additionally, if the difference between the first and second signals is greater than a determined threshold, the at least one microbe is susceptible to the at least one antimicrobial.

In addition to or alternative to performing an antimicrobial susceptibility test, the presence of an antimicrobial resistance marker and/or the absence of an antimicrobial susceptibility marker can be determined to indicate that the at least one microbe in a sample is resistant to that specific antimicrobial. In some aspects, the absence of an antimicrobial resistance marker and/or the presence of an antimicrobial susceptibility marker can indicate that the at least one microbe in a sample is susceptible to that specific antimicrobial. The detection methods described herein can be used to determine the presence or absence of an antimicrobial resistance marker or an antimicrobial susceptibility marker.

As used herein “antibiotic resistance marker” refers to a gene product, mRNA, polypeptide, polypeptide variant, or other macromolecule that confers resistance to a specific antimicrobial, such as by enzymatically cleaving the antimicrobial or specifically effluxing the antimicrobial. In some aspects, non-limiting examples of antimicrobial resistance markers include Aminocoumarin-resistant DNA topoisomerases (e.g., Aminocoumarin-resistant GyrB, ParE, ParY); Aminoglycoside acetyltransferases (e.g., AAC(1), AAC(2′), AAC(3), AAC(6′)); Aminoglycoside nucleotidyltransferases (e.g., ANT(2″), ANT(3″), ANT(4′), ANT(6), ANT(9)); Aminoglycoside phosphotransferases (e.g., APH(2″), APH(3″), APH(3′), APH(4), APH(6), APH(7″), APH(9)); 16S rRNA methyltransferases (e.g., ArmA, RmtA, RmtB, RmtC, Sgm); Class A β-lactamases (e.g., AER, BLA1, CTX-M, KPC, SHV, TEM, etc.); Class B (metallo-)β-lactamases (e.g., BlaB, CcrA, IMP, NDM, VIM, etc.); Class C β-lactamases (e.g., ACT, AmpC, CMY, LAT, PDC, etc.); Class D β-lactamases (e.g., OXA β-lactamase); mecA (methicillin-resistant PBP2); mutant porin proteins conferring antibiotic resistance; antibiotic-resistant Omp36, antibiotic-resistant OmpF, antibiotic-resistant PIB (por); genes modulating β-lactam resistance (e.g., bla (blaI, blaR1) and mec (mecI, mecR1) operons); Chloramphenicol acetyltransferase (CAT); Chloramphenicol phosphotransferase; Ethambutol-resistant arabinosyltransferase (EmbB); Mupirocin-resistant isoleucyl-tRNA synthetases (e.g., MupA, MupB); resistance markers for peptide antibiotics, including but not limited to integral membrane protein MprF; resistnace markers for phenicol, including but not limited to Cfr 23S rRNA methyltransferase; Rifampin ADP-ribosyltransferase (Arr); Rifampin glycosyltransferase; Rifampin monooxygenase; Rifampin phosphotransferase; Rifampin resistance RNA polymerase-binding proteins (e.g., DnaA, RbpA); Rifampin-resistant beta-subunit of RNA polymerase (RpoB); resistance markers against Streptogramins; Cfr 23S rRNA methyltransferase; Erm 23S rRNA methyltransferases (e.g., ErmA, ErmB, Erm(31), etc.); Streptogramin resistance ATP-binding cassette (ABC) efflux pumps (e.g., Lsa, MsrA, Vga, VgaB); Streptogramin Vgb lyase; Vat acetyltransferase; Fluoroquinolone acetyltransferase; Fluoroquinolone-resistant DNA topoisomerases; Fluoroquinolone-resistant GyrA, Fluoroquinolone-resistant GyrB, Fluoroquinolone-resistant ParC; Quinolone resistance protein (Qnr); Fosfomycin phosphotransferases (e.g., FomA, FomB, FosC); Fosfomycin thiol transferases (e.g., FosA, FosB, FosX); resistance markers against Glycopeptides, including not limited to VanA, VanB, VanD, VanR, VanS, etc.; resitance markers against Lincosamides; Cfr 23S rRNA methyltransferase; Erm 23S rRNA methyltransferases (e.g., ErmA, ErmB, Erm(31), etc.); Lincosamide nucleotidyltransferase (Lin); resistance markres aginst Linezolid; Cfr 23S rRNA methyltransferase; resisntance markers against Macrolides, such as Cfr 23S rRNA methyltransferase, Erm 23S rRNA methyltransferases (e.g., ErmA, ErmB, Erm(31), etc.); Macrolide esterases (e.g., EreA, EreB); Macrolide glycosyltransferases (e.g., GimA, Mgt, Ole); Macrolide phosphotransferases (MPH) (e.g., MPH(2′)-I, MPH(2′)-II); Macrolide resistance efflux pumps (e.g., MefA, MefE, Mel); Streptothricin acetyltransferase (sat); Sulfonamide-resistant dihydropteroate synthases (e.g., Sul1, Sul2, Sul3, sulfonamide-resistant FolP); resistance marksr against Tetracyclines; mutant porin PIB (por) with reduced permeability; Tetracycline inactivation enzyme TetX; Tetracycline resistance major facilitator superfamily (MFS) efflux pumps (e.g., TetA, TetB, TetC, Tet30, Tet31, etc.); Tetracycline resistance ribosomal protection proteins (e.g., TetM, TetO, TetQ, Tet32, Tet36, etc.); efflux pumps conferring antibiotic resistance: ABC antibiotic efflux pump (e.g., MacAB-TolC, MsbA, MsrA, VgaB, etc.); MFS antibiotic efflux pump (e.g., EmrD, EmrAB-TolC, NorB, GepA, etc.); multidrug and toxic compound extrusion (MATE) transporter (e.g., MepA); resistance-nodulation-cell division (RND) efflux pump (e.g., AdeABC, AcrD, MexAB-OprM, mtrCDE, etc.); small multidrug resistance (SMR) antibiotic efflux pump (e.g., EmrE); genes modulating antibiotic efflux (e.g., adeR, acrR, baeSR, mexR, phoPQ, mtrR, etc.). See e.g., MacAuthur et al., Antimicrob Agents Chemother. 2013 July; 57(7):3348-57, which is incorporated herein by reference. In some aspects, an antimicrobial resistance marker can include any protein, polypeptide, polypeptide variant, or other macromolecule known in the art to confer resitance to a specific antimicrobial or family of antimicrobials.

As used herein “antibiotic susceptibility marker” refers to a gene product, mRNA, polypeptide, polypeptide variant, or other macromolecule that confers susceptibility to a specific antimicrobial, especially in a domain at fashion. In some aspects, an antibiotic susceptibility marker can include any mutant or variant of one of the aforementioned antibiotic resistance markers comprising a mutation that reduces or eliminates the antibiotic resistance. In some aspects, non-limiting examples of antimicrobial susceptibility markers include RpsL and GyrA conferring sensitivity in a dominant fashion to two antibiotics, streptomycin and nalidixic acid, respectively (see e.g., Edgar et al., Appl Environ Microbiol. 2012 February; 78(3): 744-751). In some aspects, an antimicrobial susceptibility marker can include any protein, polypeptide, polypeptide variant, or other macromolecule known in the art to confer susceptibly to a specific antimicrobial or family of antimicrobials.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further aspects thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various aspects and equivalents thereof

What is claimed is:

1. A method of preparing a sample for detecting a microbe or microbe components present in the sample, the method comprising the following steps:
adding a substance to a sample suspected of comprising a microbe or microbe components;
digesting the sample under conditions promoting digestion of a microbe or microbe components in the sample; and
optionally contacting the digested microbe or microbe components with a matrix or matrix solution on a target substrate.

2. The method according to claim 1, wherein the substance comprises one or more of an antimicrobial mixture, an enzyme, a protease, or a carbohydrate-cleaving enzyme.

3. The method according to claim 2, wherein the protease is trypsin.

4. The method according to claim 1, wherein the sample is a patient sample and the detecting a microbe or microbe component comprises detecting a microbial infection in a patient.

5. The method according to claim 1, further comprising, prior to adding the substance, contacting the sample with a microbe-targeting molecule bound substrate and isolating from the sample a microbe or microbe components bound to the microbe-targeting molecule.

6. The method according to claim 5, wherein the substrate is a magnetic substrate, a fiber substrate, a polymer substrate, or ELISA plate.

7. The method according to claim 5, wherein the step of isolating comprises applying a magnet or magnetic field to the sample.

8. The method according to claim 5, wherein the step of isolating comprises washing the substrate with a fluid to remove unbound cells, biomolecules, or chemicals.

9. The method according to claim 8, wherein fluid comprises calcium.

10. The method according to claim 5, wherein the step of isolating is in accordance with a characteristic comprising at least one of size, mass, density, or charge.

11. The method according to claim 5, wherein the step of isolating comprises eluting the microbe or microbe components from the substrate.

12. The method according to claim 11, wherein the step of eluting comprises heating to a temperature of at least 70° C. with or without agitation.

13. The method according to claim 12, wherein the heating to a temperature of at least 70° C. is performed in calcium-free water.

14. The method according to claim 11, wherein the step of eluting comprises a pH treatment.

15. The method according to claim 11, wherein the step of eluting comprises treatment with a chelation agent.

16. The method according claim 15, wherein the chelating agent comprises at least one of ethylenediaminetetraacetic acid (EDTA), calcium disodium edetate (CaNa₂EDTA), ethylene glycol-bis(R-aminoethyl ether)-N,N,N′,N′-tetraacetic acid (EGTA), 1,2-bis(o-aminophenoxy)ethane-N,N,N′,N′-tetraacetic acid (BAPTA), deferoxamine mesylate salt (DFOM).

17. The method according to claim 5, wherein the step of isolating comprises concentrating the microbe or microbe components in the sample.

18. The method according to claim 17, wherein the isolated volume is less than the volume of the sample.

19. The method according to claim 2, wherein the antimicrobial mixture comprises at least one of an antibiotic mixture, an antifungal mixture, and an antiviral mixture.

20. The method according to claim 19, wherein the antibiotic mixture comprises one or more antibiotics from at least one antibiotic class comprising Cephalosporin, Glycopeptide, Cyclic lipopeptide, Aminoglycoside, Macrolide, Oxazolidinone, Fluoroquinolones, Lincosamides, and Carbapenem.

21. The method according to claim 19, wherein the antifungal mixture comprises one or more antifungals from at least one antifungal class comprising Polyenes, Azoles, Nucleoside Analog, Echinocandin, and Allylamine.

22. The method according to claim 19, wherein the antiviral mixture comprises one or more antivirals from at least one antiviral class comprising CCR5 anatonists, Fusion inhibitors, Nucleoside/Nucleotide reverse transcriptase inhibitors (NRTIs), Non-nucleoside reverse transcriptase inhibitors (NNRTIs), Nucleotide reverse transcriptase inhibitors (NtRTIs), Integrase inhibitors, Protease inhibitors, DNA polymerase inhibitors, Guanosine analogs, Interferon-alpha, M2 ion channel blockers, Nucleoside inhibitors, NS5A polymerase inhibitors, NS3/4A protease inhibitors, Neuraminidase inhibitors, Nucleoside analogs, and Direct acting antivirals (DAAs).

23. The method according to claim 19, wherein the antibiotic mixture comprises one or more of Cefepime, Vancomycin, Daptomycin, Amikacin, Erythromycin, Linezolid, Ciproflaxin, Lincomycin, and Meropenem.

24. The method according to claim 19, wherein the antifungal mixture comprises one or more of Caspofungin and Amphotericin.

25. The method according to claim 2, wherein the antimicrobial mixture comprises one or more of Cefepime, Vancomycin, Daptomycin, Amikacin, Erythromycin, Linezolid, Ciproflaxin, Lincomycin, Meropenem, Caspofungin and Amphotericin.

26. The method according to claim 19, wherein the antimicrobial mixture comprises at least one antibiotic mixture at a concentration from about 0.1 ug/mL to about 100 mg/mL.

27. The method according to claim 19, wherein the antimicrobial mixture comprises at least one antifungal mixture at a concentration from about 0.01 ug/mL to about 100 mg/mL.

28. The method according to claim 1, wherein the conditions promoting digestion include heating the sample.

29. The method according to claim 1, further comprising contacting the digested microbe or microbe components with a composition that is more acidic than the digested microbe or microbe components.

30. The method according to claim 29, wherein the composition that is more acidic than the digested microbe or microbe components is present at a volume equal to or greater than the volume of the digested microbe or microbe components.

31. The method according to claim 29, wherein the composition that is more acidic than the digested microbe or microbe components comprises trifluoroacetic acid (TFA), formic acid, or acetic acid.

32. The method according to claim 1, wherein the target substrate is evenly sprayed with matrix solution prior to analyzing microbe or microbe components to generate a homogenous layer of crystallized matrix on top of the substrate.

33. The method according to claim 1, further comprising analyzing the microbe or microbe components.

34. The method according to claim 33, wherein the analyzing step is performed using a method comprising at least one of volatile organic compound method; Raman spectroscopy; FFT (Fast-Fourier Transform); Fourier-Transform Infrared Spectroscopy (FTIR); infrared spectrometry; Nuclear Magnetic Resonance (NMR) spectrometry; chromatographic method, or mass spectrometric method.

35. The method according to claim 34, wherein the analyzing step is performed via a mass spectrometric method.

36. The method according to claim 35, wherein the mass spectrometric method comprises at least one of electron ionization, chemical ionization, electrospray ionization, atmospheric pressure chemical ionization, and matrix-assisted laser desorption ionization (MALDI-TOF MS).

37. The method according to claim 35, wherein the mass spectrometric method is automated.

38. The method according to claim 1, wherein the sample comprises blood, serum, plasma, sputum, urine, joint fluid, or any other tissue or biological sample.

39. The method according to claim 1, further comprising optionally culturing the sample prior to adding the substance.

40. The method according to claim 5, wherein the microbe-targeting molecule comprises a microbe surface-binding domain.

41. The method according to claim 40, wherein the microbe surface-binding domain comprises a mannose-binding lectin (MBL).

42. The method according to claim 41, wherein the microbe surface-binding domain comprises a human mannose-binding lectin (MBL).

43. The method according to claim 40, wherein the microbe surface-binding domain comprises a carbohydrate recognition domain (CRD) of MBL.

44. The method according to claim 43, wherein the CRD is linked to an immunoglobulin or fragment thereof.

45. The method according to claim 43, wherein the CRD is linked to an Fc component of human IgG1 (FcMBL).

46. The method according to claim 6, wherein the magnetic substrate is a superparamagnetic substrate.

47. The method according to claim 6, wherein the magnetic substrate comprises at least one of a magnetic bead, a superparamagnetic bead, or a magnetic microbead.

48. The method according to claim 5, wherein the microbe-targeting molecule is linked to an ELISA plate.

49. The method according to claim 1, wherein the microbe comprises a Gram-positive bacterial species, a Gram-negative bacterial species, a mycobacterium, a fungus, a parasite, a bacterial antigen, a viral antigen, a protozoan, an alga, or a virus.

50. The method according to claim 1, wherein the microbe component comprises a component from a Gram-positive bacterial species, a Gram-negative bacterial species, a mycobacterium, a fungus, a parasite, a bacterial antigen, a viral antigen, a protozoan, an alga, or a virus.

51. The method according to claim 1, wherein the microbe component comprises microbe-associated molecular patterns (MAMPs) and/or microbe-associated proteins.

52. The method according to claim 1, wherein the microbe is a pathogen that affects humans.

53. The method according to claim 1, wherein the microbe or microbe component is a pathogen or component thereof.

54. The method according to claim 53, further comprising identifying the group of the pathogen.

55. The method according to claim 53, further comprising identifying the domain of the pathogen.

56. The method according to claim 53, further comprising identifying the species of the pathogen.

57. The method according to claim 53, further comprising identifying the strain of the pathogen.

58. The method according to 53, further comprising identifying the antimicrobial susceptibility of the pathogen.

59. A method of preparing a sample for detecting a microbe or microbe components present in the sample, the method comprising the following steps:
isolating from a sample a microbe or microbe components bound to a microbe-targeting molecule on a substrate;
adding a substance to the isolated microbe or microbe components;
digesting the isolated microbe or microbe components under conditions promoting digestion of the microbe or microbe components; and
contacting the digested microbe or microbe components with a matrix or matrix solution on a target substrate.
